(19) United States
(12) Patent Application Publication
(10) Pub. No.: US 2008/0027096 A1
(43) Pub. Date: Jan. 31, 2008

(54) Novel Pharmaceutical Compositions

(76) Inventors: Neeraj Garg, Tumba (SE); Eva Kristina Kock, Stockholm (SE); Henrik Per Hakan Jernstedt, Uppsala (SE); Mikael Johan Gillner, Stockholm (SE)

Correspondence Address: WIGGIN AND DANA LLP;ATTENTION: PATENT DOCKETING, ONE CENTURY TOWER, P.O. BOX 1832, NEW HAVEN, CT, 06508-1832, US

(21) Appl. No.: 10/591,621

(22) PCT Filed: Mar. 4, 2005
(86) PCT No.: PCT/EP05/02307

§ 371 Date: Jul. 9, 2007

(30) Foreign Application Priority Data
Mar. 5, 2004 (GB) 0405033.2

(51) Int. Cl. A61K 31/19; A61K 31/35; A61K 31/381; A61K 31/42; A61K 31/426; A61K 31/435; A61K 31/47; A61P 3/00; C07C 69/95; C07D 211/70; C07D 215/00; C07D 261/06; C07D 277/22; C07D 315/00; C07D 333/56; G01N 33/566

(52) U.S. Cl. 514/311; 436/501; 514/277; 514/365; 514/378; 514/443; 514/451; 514/568; 546/152; 546/339; 548/203; 548/247; 549/427; 549/58; 560/21; 560/55

(57) ABSTRACT

The invention provides compounds of formula (I) or a pharmaceutically acceptable ester, amide, solvate or salt thereof, including a salt of such an ester or amide, and a solvate of such an ester, amide or salt, for use in the treatment or prophylaxis of condition mediated by an androgen receptor.

### FIELD OF THE INVENTION

The present invention relates to compounds which are antagonists, partial antagonists or partial agonists to the action of endogenous hormones, for example testosterone or dihydrotestosterone, on the androgen receptor and the use of such compounds for therapeutic purposes

### BACKGROUND OF THE INVENTION

The androgen receptor (AR) is a member of the steroid hormone nuclear receptor family of ligand activated transcription factors. The family includes estrogen, progesterone, mineralocorticoid, and glucocorticoid receptors, all of which are activated by endogenous steroid hormones to control the expression of responsive genes. The hormone receptors share a modular structure consisting of a variable amino-terminal domain (NTD), a highly conserved DNA-binding domain (DBD), and a carboxy-terminal ligand-binding domain (LBD). The DNA-binding domain generates much of the transcriptional specificity due to its ability to discern different DNA response elements with the promoter regions of target genes. The LBD is required for ligand-dependent transcriptional activity and it contains both the hormone-binding pocket and an important transcriptional activation functional region (AF2) required for recruitment of coactivators and the cellular transcriptional machinery.

Nuclear receptor activity is regulated predominantly by the binding of the hormone ligand within the LBD. The amino acids lining the interior of the hormone-binding cavity define the selectivity of the receptor for its hormone. This allows the AR to discriminate between the natural ligands and non-natural ligands.

The natural ligand for the androgen receptor, androgen, is produced in both men and women by the gonads, adrenal glands and locally in target tissues. The levels of androgens secreted by the gonads are tightly regulated by a feedback mechanism involving the hypothalamus and pituitary.

In men, androgens are necessary for masculinization and fertility. However, systemic androgen excess causes testicular atrophy and infertility. Androgen excess may also contribute to cardiovascular disease and psychological abnormalities, including, but not limited to, mood (for example aggression and anxiety), see for example Clark, A. et al., Neurosci Biobehav. Rev., 2003, 27(5), 413-436. Local androgen excess is implicated in the pathogenesis of male pattern baldness (alopecia), benign prostatic hyperplasia (BPH) and acne. The physiological role of androgens in women is not well understood, but they have been found to play a role in the development of normal body hair and libido. In women, relative androgen excess causes hirsutism (excessive hair growth), amenorrhea (abnormal loss or suppression of menses), acne and male pattern baldness. Androgen deficit may contribute to cardiovascular disease and psychological abnormalities, including, but not limited to, mood (for example depression) and cognitive function, see for example the side effect conditions discussed in Chen, A and Petrylak D. (Current Oncology Reports, 2004, 6, 209-215).

The risk of developing prostate cancer increases dramatically with age. More than 75% of prostate cancer diagnoses are in men over the age of 65, and the prevalence of clinically undetectable prostate cancer in men over 80 years old is as high as 80%. The exact cause of prostate cancer remains unclear. It is, however, widely accepted that androgens can increase the severity and the rate of progression of the disease. Androgen deprivation therapy has been the basis for prostate cancer therapy since 1941 when castration was shown to have beneficial effects on advanced stages of the disease. Hormonal intervention is currently based on disrupting the hypothalamus-pituitary-gonadal feedback mechanism to control the levels of endogenous androgens from the testes. Antiandrogens are incorporated in later stage therapies to work at the level of the androgen receptor itself, blocking residual androgens from adrenal sources. In spite of these treatments, there exists a need for an improved therapy of diseases linked to disturbances in the activity of the androgen receptor.

### SUMMARY OF THE INVENTION

The present invention provides a compound of formula (I) or a pharmaceutically acceptable ester, amide, solvate or salt thereof, including a salt of such an ester or amide, and a solvate of such an ester, amide or salt, for use in the treatment or prophylaxis of a condition mediated by an androgen receptor,

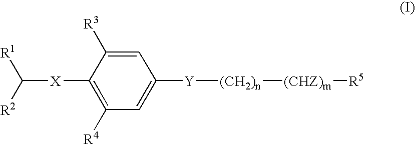

wherein:
R¹ is selected from C₅₋₁₀ aryl, C(O)—C₅₋₁₀ aryl, C(O)—C₃₋₈ heterocyclyl, C₅₋₁₀ aryl-C₁₋₂ alkyl, C₃₋₁₀ heterocyclyl, C₃₋₁₀ heterocyclyl-C₁₋₂ alkyl, C₃₋₁₅ alkyl, C₄₋₁₅ alkenyl, C₃₋₁₅ alkynyl, C₃₋₁₀ cycloalkyl and C₃₋₁₀ cycloalkyl C₁₋₂ alkyl, said alkyl, alkenyl and alkynyl groups or portions of groups optionally being substituted with, where applicable, 1 to 3 groups R^{a} which may be the same or different; said heterocyclyl and cycloalkyl groups or portions of groups optionally being substituted with, where applicable, 1 to 3 groups R^{a′} which may be the same or different; said aryl groups or portions of groups optionally being substituted with, where applicable, 1 to 4 groups R^{a} which may be the same or different;

### R² is selected from hydrogen, C₁₋₄ alkyl, C₂₋₄ alkenyl, C₂₋₄ alkynyl and C₁₋₄ alkoxy;

or R¹ and R² together with the carbon atom to which they are both attached form a C₄₋₈ cycloalkyl, C₄₋₈ cycloalkenyl, a saturated or partially saturated C₃₋₁₀ heterocyclyl, optionally substituted with, where applicable, 1 to 3 groups R^{a} which may be the same or different;

### X is selected from CH₂, oxygen, sulfur, sulfoxide, sulfone, selenium, tellurium, disulfide, and a group of formula —N(R^{c})—;

R³ and R⁴ are independently selected from hydrogen, halogen, C₁₋₄ alkyl, C₃₋₇ cycloalkyl, C₃₋₇ heterocyclyl, C₂₋₄ alkenyl, C₂₋₄ alkynyl, C₁₋₄ alkoxy, fluoromethyl, difluoromethyl, trifluoromethyl, fluoromethoxy, difluoromethoxy, trifluoromethoxy, methylthio, fluoromethylthio, difluoromethylthio, trifluoromethylthio, and COOR^{c};

### Y is selected from bond, carbonyl, oxygen, sulphur, —CH(R^{b})—, —NHCO—, —CONH—, —NHSO₂—, —SO₂NH—, —NR^{c})— and —CR⁶═CR⁷—;

n is selected from 0, 1, 2 and 3;

### Z is selected from halogen, amino, hydroxy, mercapto, C₁₋₄ alkyl, C₂₋₄ alkenyl, C₂₋₄ alkynyl, C₁₋₄ alkoxy, fluoromethyl, difluoromethyl, trifluoromethyl, fluoromethoxy, difluoromethoxy, trifluoromethoxy and (CH₂)ₚOH, where p is an integer from 1 to 4;

m is selected from 0 and 1;

### R⁵ is selected from —CO₂R^{c}, —PO(OR^{c})_{2,} —PO(OR^{c})NH₂, —SO₂OR^{c}, —COCO₂R^{c}, CONR^{c}OR^{c}, —SO₂NHR^{c}, —NHSO₂R^{c}′, —CONHSO₂R^{c}′, and —SO₂NHCOR^{c};

R⁶ and R⁷ are independently selected from hydrogen, halogen, C₁₋₄ alkyl, C₂₋₄ alkenyl, C₂₋₄ alkynyl, C₁₋₄ alkoxy, C₅₋₁₀ aryl, fluoromethyl, difluoromethyl, trifluoromethyl, fluoromethoxy, difluoromethoxy, trifluoromethoxy, and (CH₂)ₚOH, where p is an integer from 1 to 4;

### R^{a} is selected from halogen, C₁₋₄ alkoxy, C₅₋₁₀ aryl, fluoromethoxy, difluoromethoxy, trifluoromethoxy, methylthio, fluoromethylthio, difluoromethylthio, trifluoromethylthio, mercapto, cyano, and nitro;

### R^{a′} is selected from R^{a}, fluoromethyl, difluoromethyl, trifluoromethyl, C₁₋₄ alkyl, C₃₋₁₀ heterocyclyl-C₂₋₄ alkenyl, C₅₋₁₀ aryl-C₂₋₄ alkenyl, C₃₋₁₀ heterocyclyl-C₁₋₄ alkyl and C₅₋₁₀ aryl-C₁₋₄ alkyl

R^{a″} is selected from:
R^{a′},
C₂₋₄ alkenyl, optionally substituted with 1, 2 or 3 groups selected from C₅₋₁₀ aryl, C(O)R^{c}, C₃₋₁₀ heterocyclyl, and C₃₋₁₀ heterocyclyl substituted with C₁₋₄ alkyl;
C₂₋₈ alkenyloxy;
C₃₋₈ cycloalkyl-C₁₋₃ alkoxy, C₅₋₁₀ aryl-C₁₋₃ alkoxy, and C₅₋₁₀ aryloxy, said C₃₋₈ cycloalkyl-C₁₋₃ alkoxy, C₅₋₁₀ aryl-C₁₋₃ alkoxy or C₅₋₁₀ aryloxy optionally being substituted with 1, 2 or 3 groups selected from C₁₋₄ alkyl, halogen, fluoromethyl, difluoromethyl, trifluoromethyl, fluoromethoxy, difluoromethoxy, trifluoromethoxy, methylthio, mercapto, hydroxy, cyano, nitro, a group of formula -N(R^{c})₂ in which the two R^{c} groups may be the same or different but not both simultaneously hydrogen;
   R^{b} is selected from hydrogen, halogen, hydroxyl, mercapto, C₁₋₄ alkyl, C₂₋₄ alkenyl, C₂₋₄ alkynyl, C₁₋₄ alkoxy, fluoromethyl, difluoromethyl, trifluoromethyl, fluoromethoxy, difluoromethoxy, trifluoromethoxy and (CH₂)ₚOH, where p is an integer from 1 to 4; and

### R^{c} is selected from hydrogen, C₁₋₄ alkyl, C₂₋₄ alkenyl and C₂₋₄ alkynyl; and

### R^{c′} is selected from R^{c}, C₅₋₁₀ aryl and C₅₋₁₀ aryl substituted with 1, 2 or 3 groups selected from amino, hydroxy, halogen or C₁₋₄ alkyl.

Compounds of the invention have surprisingly been found to be antagonists, partial antagonists or partial agonists to the action of endogenous hormones, for example testosterone or dihydrotestosterone, at the androgen receptor. Preferred compounds of the invention are antagonists or partial antagonists of the androgen receptor.

### DETAILED DESCRIPTION OF THE INVENTION

The compounds of formula (I) may contain chiral (asymmetric) centres, or the molecule as a whole may be chiral. The individual stereoisomers (enantiomers and diastereoisomers) and mixtures of these are within the scope of the present invention.

Preferably, R¹ is selected from C₅₋₁₀ aryl, C(O)—C₅₋₁₀ aryl, C(O)—C₃₋₈ heterocyclyl, C₃₋₁₀ heterocyclyl, C₅₋₁₀ heterocyclyl-C₁₋₂-alkyl, C₃₋₁₅ alkyl and C₄₋₈ cycloalkyl, said alkyl groups or portions of groups optionally being substituted with, where applicable 1 to 3 groups R^{a} which may be the same or different; said heterocyclyl and cycloalkyl groups or portions of groups optionally being substituted with, where applicable, 1 to 3 groups R^{a} which may be the same or different; said aryl groups or portions of groups optionally being substituted with, where applicable, 1 to 4 groups R^{a″} which may be the same or different.

More preferably, R¹ is selected from C₆₋₁₀ aryl, C(O)—C₆₋₁₀ aryl, C(O)—C₃₋₈ heterocyclyl C₅₋₁₀ heterocyclyl-C₁₋₂-alkyl, C₄₋₁₀ alkyl and C₅₋₇ cycloalkyl, said alkyl groups or portions of groups optionally being substituted with, where applicable 1 to 3 groups R^{a} which may be the same or different; said heterocyclyl and cycloalkyl groups or portions of groups optionally being substituted with, where applicable, 1 to 3 groups R^{a′} which may be the same or different; said aryl groups or portions of groups optionally being substituted with, where applicable, 1 to 4 groups R^{a″} which may be the same or different.

More preferably, R¹ is selected from phenyl or branched C₄₋₁₀ alkyl, said alkyl optionally being substituted with, where applicable 1 to 3 groups R^{a} which may be the same or different, said phenyl optionally being substituted with, where applicable, 1 to 3 groups R^{a″} which may be the same or different.

Most preferably, R¹ is phenyl, preferably substituted with 1, 2 or 3 groups R^{a″} which may be the same or different. Preferred locations for the R^{a″} group or groups are the 2- or 3-position relative to the attachment point to the —CH(R²)X— of the remainder of the molecule.

In a particularly preferred embodiment, R¹ is phenyl, substituted with three methyl groups in the 2, 4 and 6 positions. In an alternative preferred embodiment, R¹ is phenyl, substituted with a difluoromethoxy or a trifluoromethoxy group in the 2 position.

R² is preferably selected from hydrogen, C₁₋₂ alkyl, C₂₋₃ alkenyl, C₂₋₃ alkynyl and C₁₋₂ alkoxy.

More preferably, R² is selected from hydrogen, methyl or methoxy. Most preferably, R² is hydrogen.

In an alternative preferred embodiment, R¹ and R² together with the carbon atom to which they are both attached form a C₄₋₈ cycloalkyl group or a saturated C₃₋₈ heterocyclyl group, optionally substituted with, where applicable, 1 to 3 groups R^{a} which may be the same or different. In such an embodiment, preferably, R¹ and R² together with the carbon atom to which they are both attached form a C₅₋₇ cycloalkyl group or a saturated C₃₋₆ heterocyclyl group, most preferably, a cyclohexyl or a tetrahydrofuranyl group.

Preferably, X is selected from oxygen, sulfur and sulfoxide. More preferably, X is sulfur or oxygen, most preferably oxygen.

R³ and R⁴ are preferably independently selected from hydrogen, halogen, C₁₋₂ alkyl, C₁₋₂ alkoxy, fluoromethyl, difluoromethyl, trifluoromethyl, fluoromethoxy, difluoromethoxy and trifluoromethoxy. More preferably R³ and R⁴ are independently selected from halogen, C₁₋₂ alkyl, C₁₋₂ alkoxy, fluoromethyl, difluoromethyl, trifluoromethyl, fluoromethoxy, difluoromethoxy and trifluoromethoxy.

More preferably, R³ and R⁴ are preferably independently selected from halogen and C₁₋₂ alkoxy. Amongst the halogens, there are preferred bromine, chlorine and fluorine, especially bromine and chlorine, in particular bromine.

R³ and R⁴ may simultaneously represent the same radical. Alternatively, R³ and R⁴ are different from each other. It is preferred that l³ and R⁴ are not both simultaneously hydrogen. In an alternative embodiment, R³ and R⁴ are both simultaneously hydrogen.

Y is preferably selected from bond, carbonyl, oxygen, sulphur, —CH(R^{b})—, —NHCO—, —NHSO₂—, —SO₂NH—, —N(R^{c})— and —CR⁶═CR⁷—; Y is more preferably selected from oxygen, carbonyl and —CH(R^{b})—. Most preferably Y is selected from carbonyl and CH₂.

n is preferably 0, 1 or 2; more preferably, n is 0 or 1, for example 1.

When m=1, Z is preferably selected from halogen and hydroxy. More preferably, Z is bromine, chlorine or hydroxyl.

Alternatively, m may be 0.

R⁵ is preferably selected from —CO₂R^{c}, —PO(OR^{c})₂, —SO₂OR^{c}, —NHSO₂R^{c′}, —COCO₂R^{c} and CONR^{c}OR^{c}. More preferably, R⁵ is —CO₂R^{c}, —PO(OR^{c})₂ or —SO₂OR^{c}. Most preferably, R⁵ is —CO₂R^{c}, particularly —CO₂H.

R⁶ and R⁷ are preferably independently selected from hydrogen, C₁₋₄ alkyl, C₂₋₄ alkenyl and C₁₋₄ alkoxy. More preferably, R⁶ and R⁷ are independently selected from hydrogen, methyl and methoxy. Most preferably R⁶ and R⁷ are hydrogen.

When R^{a} or R^{a′} is a halogen, it is preferably selected from bromine, chlorine and fluorine, especially bromine. Substitution with two or three halogen groups is, in some circumstances, preferred. When the basic structure of R¹ includes a CH₃ or OCH₃ group, appropriate substitution of that group may lead to a difluoromethyl, a trifluormethyl, a difluoromethoxy or a trifluormethoxy group being present in the molecule.

Other preferred selections for R^{a} are fluoromethoxy, difluoromethoxy, trifluoromethoxy and hydroxyl groups. More preferably, R^{a} is selected from difluoromethoxy and trifluoromethoxy.

R^{a′} is preferably R^{a} or C₁₋₄ alkyl, fluoromethyl, difluoromethyl, trifluoromethyl. Most preferably, R^{a′} is R^{a}, C₁₋₂ alkyl or trifluoromethyl.

Preferably, R^{a′} is selected from R^{a}, fluoromethyl, difluoromethyl, trifluoromethyl, C₁₋₄ alkyl, and C₃₋₁₀ heterocyclyl-C₂₋₄ alkenyl.

R^{a″} is preferably R^{a′}.

When R¹ includes an aryl group, there are preferably 1 to 3 groups R^{a″} present in the molecule.

R^{b} is preferably selected from hydrogen, C₁₋₄ alkyl, fluoromethyl, difluoromethyl and trifluoromethyl. More preferably, R^{b} is selected from hydrogen and C₁₋₂ alkyl. Most preferably, R^{b} is hydrogen.

R^{c} is preferably selected from hydrogen and C₁₋₂ alkyl. More preferably, R^{c} is selected from hydrogen and methyl, particularly hydrogen.

Accordingly, one preferred group of compounds of the invention includes compounds according to formula (Ia) or a pharmaceutically acceptable ester, amide, solvate or salt thereof, including a salt of such an ester or amide, and a solvate of such an ester, amide or salt

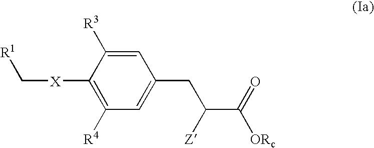

wherein:
R¹ is selected from C₆₋₁₀ aryl, C₅₋₁₀ heterocyclyl-C₁₋₂-alkyl, C₄₋₁₀ alkyl and C₅₋₇ cycloalkyl, said alkyl optionally being substituted with, where applicable, 1 to 3 groups R^{a} which may be the same or different; said cycloalkyl optionally being substituted with, where applicable, 1 to 3 groups R^{a} which may be the same or different; and said aryl optionally being substituted with, where applicable, 1 to 3 groups R^{a″} which may be the same or different;

### X is selected from oxygen and sulfur;

### R³ and R⁴ are independently selected from hydrogen, halogen, C₁₋₂ alkyl, C₁₋₂ alkoxy, fluoromethyl, difluoromethyl, trifluoromethyl, fluoromethoxy, difluoromethoxy and trifluoromethoxy;

### Z′ is selected from hydrogen, halogen, hydroxyl and mercapto;

### R^{a} is selected from halogen, C₅₋₁₀ aryl , fluoromethoxy, difluoromethoxy, trifluoromethoxy and nitro;

### R^{a′} is selected from R^{a}, fluoromethyl, difluoromethyl, trifluoromethyl, C₁₋₄ alkyl, C₅₋₁₀ heterocyclyl-C₂₋₄ alkenyl, C₅₋₁₀ aryl-C₂₋₄ alkenyl, C₅₋₁₀ heterocyclyl-C₁₋₄ alkyl and C₅₋₁₀ aryl-C₂₋₄ alkyl;

R^{a″} is selected from:
R^{a′};
C₂₋₄ alkenyl, substituted with C₃₋₁₀ heterocyclyl;
C₅₋₁₀ aryloxy, optionally being substituted with 1, 2 or 3 groups selected from C₁₋₄ alkyl, halogen, fluoromethyl, difluoromethyl, trifluoromethyl, fluoromethoxy, difluoromethoxy, trifluoromethoxy, methylthio, mercapto, hydroxy, cyano, or nitro;
   and

### R^{c} is selected from hydrogen and C₁₋₄ alkyl.

Preferred compounds according to the invention include:
1: (3,5-dibromo-4-sec-butoxyphenyl)acetic acid
2: (3,5-dibromo-4-isobutoxyphenyl)acetic acid
3: [3,5-dibromo-4-(pentyloxy)phenyl]acetic acid
4: [3,5-dibromo-4-(3-methylbutoxy)phenyl]acetic acid
5: [3,5-dibromo-4-(hexyloxy)phenyl]acetic acid
6: [3,5-dibromo-4-(2-ethylbutoxy)phenyl]acetic acid
7: [3,5-dibromo-4-(cyclohexylmethoxy)phenyl]acetic acid
8: 3-(3,5-dibromo-4-sec-butoxyphenyl)propanoic acid
9: 3-[3,5-dibromo-4-(pentyloxy)phenyl]propanoic acid
10: 3-[3,5-dibromo-4-(hexyloxy)phenyl]propanoic acid
11: 3-[3,5-dibromo-4-(3-methylbutoxy)phenyl]propanoic acid
12: 3-[3,5-dibromo-4-(2-ethylbutoxy)phenyl]propanoic acid
13: 3-[3,5-dibromo-4-(cyclohexylmethoxy)phenyl]propanoic acid
14: 3-[3,5-dibromo-4-(3-cyclohexylpropoxy)phenyl]propanoic acid
15: 3-{3,5-dibromo-4-[(3-methylbenzyl)oxy]phenyl}propanoic acid
16: 3-{3,5-dibromo-4-[(2Z)-pent-2-en-1-yloxy]phenyl}propanoic acid
17: 3-{3,5-dibromo-4-[(3-methylbenzyl)oxy]phenyl}propanoic acid
18: 3-[3,5-dibromo-4-(pent-4-en-1-yloxy)phenyl]propanoic acid
19: 3-[3,5-dibromo-4-(but-2-yn-1-yloxy)phenyl]propanoic acid
20: (3,5-dibromo-4-butoxyphenyl)acetic acid
21: 3-{3,5-dibromo-4-[(3-methylbenzyl)oxy]phenyl}acetic acid
22: 3-(3,5-dibromo-4-{[3-(trifluoromethyl)benzyl]oxy}phenyl)propanoic acid
23: 3-{3,5-dibromo-4-[(3-bromobenzyl)oxy]phenyl}propanoic acid
24: 3-{3,5-dibromo-4-[(2-methylbenzyl)oxy]phenyl}propanoic acid
25: 3-{3,5-dibromo-4-[(4-methylbenzyl)oxy]phenyl}propanoic acid
26: 3-{3,5-dibromo-4-[(3,5-dimethylbenzyl)oxy]phenyl}propanoic acid
27: 3-{3,5-dibromo-4-[(4-fluorobenzyl)oxy]phenyl}propanoic acid
28: 3-(3,5-dibromo-4-{[4-(trifluoromethyl)benzyl]oxy}phenyl)propanoic acid
29: 3-{3,5-dibromo-4-[(3-nitrobenzyl)oxy]phenyl}propanoic acid
30: 3-{3,5-dibromo-4-[(4-tert-butylbenzyl)oxy]phenyl}propanoic acid
31: N-[3,5-Dibromo-4-(3-bromobenzyloxy)benzoyl]benzenesulphonamide
32: N-[3,5-Dibromo-4-(3-bromobenzyloxy)benzoyl]-3-nitrobenzenesulphonamide
33: N-[3,5-Dibromo-4-(3-bromobenzyloxy)benzoyl]-4-nitrobenzenesulphonamide
34: 4-Amino-N-[3,5-dibromo-4-(3-bromobenzyloxy)benzoyl]benzenesulphonamide
35: N-[3,5-Dibromo-4-(3-bromobenzyloxy)benzoyl]methanesulphonamide
36: {3,5-dibromo-4-[(2-ethylhexyl)oxy]phenyl}acetic acid
37: [3,5-dibromo-4-(cyclopropylmethoxy)phenyl]acetic acid
38: 3-[3,5-dibromo-4-(2-naphthylmethoxy)phenyl]propanoic acid
39: 3-{3,5-dibromo-4-[(3,5-dibromobenzyl)oxy]phenyl}propanoic acid
40: 3-{3,5-dibromo-4-[(3-cyanobenzyl)oxy]phenyl}propanoic acid
41: 3-{3,5-dibromo-4-[(3 -methoxybenzyl)oxy]phenyl}propanoic acid
42: (4-butoxy-3,5-dichlorophenyl)(hydroxy)acetic acid
43: [3,5-dichloro-4-(heptyloxy)phenyl](hydroxy)acetic acid
44: {4-[(3-bromobenzyl)oxy]-3,5-dichlorophenyl}(hydroxy)acetic acid
45: (4-butoxy-3,5-dichlorophenyl)(oxo)acetic acid
46: [3,5-dichloro-4-(heptyloxy)phenyl](oxo)acetic acid
47: {4-[(3-bromobenzyl)oxy]-3,5-dichlorophenyl}(oxo)acetic acid
48: {3,5-dichloro-4-[(3,5-dimethylbenzyl)oxy]phenyl}(oxo)acetic acid
49: {3,5-dichloro-4-[(3-methoxybenzyl)oxy]phenyl}(oxo)acetic acid
50: (4-butoxy-3,5-dimethylphenyl)acetic acid
51: [4-(cyclohexylmethoxy)-3,5-dimethylphenyl]acetic acid
52: [4-(heptyloxy)-3,5-dimethylphenyl]acetic acid
53: {4-[(3-bromobenzyl)oxy]-3,5-dimethylphenyl}acetic acid
54: {4-[(3,5-dimethylbenzyl)oxy]-3,5-dimethylphenyl}acetic acid
55: {4-[(3-methoxybenzyl)oxy]-3,5-dimethylphenyl}acetic acid
56: (4-butoxy-3,5-dimethylphenyl)(oxo)acetic acid
57: [4-(heptyloxy)-3,5-dimethylphenyl](oxo)acetic acid
58: {4-[(3-bromobenzyl)oxy]-3,5-dimethylphenyl}(oxo)acetic acid
59: {4-[(3,5-dimethylbenzyl)oxy]-3,5-dimethylphenyl}(oxo)acetic acid
60: {4-[(3 -methoxybenzyl)oxy]-3,5-dimethylphenyl}(oxo)acetic acid
61: (4-butoxy-3,5-diisopropylphenyl)(oxo)acetic acid
62: {4-[(2-ethylhexyl)oxy]-3,5-diisopropylphenyl}(oxo)acetic acid
63: [4-(cyclohexylmethoxy)-3,5-diisopropylphenyl](oxo)acetic acid
64: [4-(heptyloxy)-3,5-diisopropylphenyl](oxo)acetic acid
65: {4-[(3-bromobenzyl)oxy]-3,5-diisopropylphenyl}(oxo)acetic acid
66: {4-[(3,5-dimethylbenzyl)oxy]-3,5-diisopropylphenyl}(oxo)acetic acid
67: {3,5-diisopropyl-4-[(3-methoxybenzyl)oxy]phenyl}(oxo)acetic acid
68: (4-butoxy-3,5-diisopropylphenyl)acetic acid
69: {4-[(3-bromobenzyl)oxy]-3,5-diisopropylphenyl}acetic acid
70: {4-[(3,5-dimethylbenzyl)oxy]-3,5-diisopropylphenyl}acetic acid
71: {3,5-diisopropyl-4-[(3-methoxybenzyl)oxy]phenyl}acetic acid
72: 3-(3,5-dibromo-4-{[3-(trifluoromethoxy)benzyl]oxy}phenyl)propanoic acid
73: 3-(3,5-dibromo-4-{[3-fluoro-5-(trifluoromethyl)benzyl]oxy}phenyl)propanoic acid
74: 3-{3,5-dibromo-4-[(3-fluorobenzyl)oxy]phenyl}propanoic acid
75: 3-{3,5-dibromo-4-[(3,5-difluorobenzyl)oxy]phenyl}propanoic acid
76: 3-(4-{[3,5-bis(trifluoromethyl)benzyl]oxy}-3,5-dibromophenyl)propanoic acid
77: 3-(3,5-dibromo-4-{[3-(difluoromethoxy)benzyl]oxy}phenyl)propanoic acid
78: 3-[3,5-dibromo-4-(1-naphthylmethoxy)phenyl]propanoic acid
79: 3-[3,5-dibromo-4-(pyridin-2-ylmethoxy)phenyl]propanoic acid
80: 3-[3,5-dibromo-4-(pyridin-3-ylmethoxy)phenyl]propanoic acid
81: 3-[3,5-dibromo-4-(pyridin-4-ylmethoxy)phenyl]propanoic acid
82: 3-[3,5-dibromo-4-(quinolin-2-ylmethoxy)phenyl]propanoic acid
83: 3-{3,5-dibromo-4-[(5-chloro-1-benzothien-3-yl)methoxy]phenyl}propanoic acid
84: 3-(3,5-dibromo-4-{[4-chloro-2-(trifluoromethyl)quinolin-6-yl]methoxy}phenyl)-propanoic acid
85: 3-{3,5-dibromo-4-[(5-methylisoxazol-3-yl)methoxy]phenyl}propanoic acid
86: 3-{3,5-dibromo-4-[(3,5-dichlorobenzyl)oxy]phenyl}propanoic acid
87: 3-{3,5-dibromo-4-[(2-fluorobenzyl)oxy]phenyl}propanoic acid
88: 3-[3,5-dibromo-4-(mesitylmethoxy)phenyl]propanoic acid
89: 3-{3,5-dibromo-4-[(2-methyl-1,3-thiazol-4-yl)methoxy]phenyl}propanoic acid
90: 3-{3,5-dibromo-4-[(3-chlorobenzyl)oxy]phenyl}propanoic acid
91: 3-{3,5-dibromo-4-[(2-chlorobenzyl)oxy]phenyl}propanoic acid
92: 3-{3,5-dibromo-4-[(3-iodobenzyl)oxy]phenyl}propanoic acid
93: 3-[3,5-dibromo-4-(tetrahydro-2H-pyran-2-ylmethoxy)phenyl]propanoic acid
94: 3-{3,5-dibromo-4-[(2-nitrobenzyl)oxy]phenyl}propanoic acid
95: 3-(3,5-dibromo-4-{[2-(difluoromethoxy)benzyl]oxy}phenyl)propanoic acid
96: 3-(3,5-dibromo-4-{[2-(trifluoromethoxy)benzyl]oxy}phenyl)propanoic acid
97: 3-{3,5-dibromo-4-[(1-bromo-6-fluoro-2-naphthyl)methoxy]phenyl}propanoic acid
98: 3-(3,5-dibromo-4-{[2-(trifluoromethyl)benzyl]oxy}phenyl)propanoic acid
99: 3-{3,5-dibromo-4-[(1-bromo-2-naphthyl)methoxy]phenyl}propanoic acid
100: 3-{3,5-dibromo-4-[(6-methoxy-2-naphthyl)methoxy]phenyl}propanoic acid
101: 3-[3,5-dibromo-4-(3-ethoxybenzyloxy)phenyl]propionic acid
102: 3 -[3,5-dibromo-4-(3-propyloxybenzyloxy)phenyl]propionic acid
103: 3-[3,5-dibromo-4-(3-butyloxybenzyloxy)phenyl]propionic acid
104: 3-[3,5-dibromo-4-(3-aminobenzyloxy)phenyl]propionic acid
106: 3-[3,5-dibromo-4-(3-diethylaminebenzyloxy)phenyl]propionic acid
107: N-[3,5-Dibromo-4-(3-bromobenzyloxy)phenyl]oxamic acid
108: N-[3,5-Dibromo-4-(2-methylnaphthyloxy)phenyl]oxamic acid
109: 3-[3-bromo-5-methoxy-4-(3-bromobenzyloxy)phenyl]propionic acid
110: 3-[3-bromo-5-methoxy-4-(2-methylnaphthyloxy)phenyl]propionic acid
111: 3-(3,5-dibromo-4-{[4-(trifluoromethyl)benzyl]oxy}phenyl)acrylic acid
112: 3-{3,5-dibromo-4-[(2-methylbenzyl)oxy]phenyl}acrylic acid
113: 3-(3,5-dibromo-4-{[3-(trifluoromethoxy)benzyl]oxy}phenyl)acrylic acid
114: 3-[3,5-dibromo-4-(pyridin-2-ylmethoxy)phenyl]acrylic acid
115: 3-[3,5-dibromo-4-(quinolin-2-ylmethoxy)phenyl]acrylic acid
116: 3-(3,5-dibromo-4-{[3-fluoro-5-(trifluoromethyl)benzyl]oxy}phenyl)acrylic acid
117: 3-{3,5-dibromo-4-[(5-methylisoxazol-3-yl)methoxy]phenyl}acrylic acid
118: 3-{3,5-dibromo-4-[(3-bromobenzyl)oxy]phenyl}acrylic acid
119: 3-[3,5-dibromo-4-(2-naphthylmethoxy)phenyl]acrylic acid
120: 3-{3-bromo-4-[(3-bromobenzyl)oxy]-5-methoxyphenyl}acrylic acid
121: 3-[3-bromo-5-methoxy-4-(2-naphthylmethoxy)phenyl]acrylic acid
122: 3-{3,5-dibromo-4-[2-(1H-indol-3-yl)ethoxy]phenyl}propanoic acid
123: 3-[4-(biphenyl-2-ylmethoxy)-3,5-dibromophenyl]propanoic acid
124: 3-{3,5-dibromo-4-[(3-bromobenzyl)oxy]phenyl}-2-hydroxypropanoic acid
125: 2-chloro-3-{3,5-dibromo-4-[(3-bromobenzyl)oxy]phenyl}propanoic acid
126: 3-[3,5-dibromo-4-({2-[(E)-2-pyridin-4-ylvinyl]benzyl}oxy)phenyl]propanoic acid

The following compounds are also preferred:
127: 3-(3,5-dibromo-4-{[3-(4-fluorophenoxy)benzyl]oxy}phenyl)propanoic acid
128: 3-(3,5-dibromo-4-{[3-(2-phenyl(E)vinyl)benzyl]oxy}phenyl)propanoic acid
129: 3-[3,5-dibromo-4-({3-[(E)-2-(4-methyl-1,3-thiazol-5-yl)vinyl]benzyl}oxy)phenyl] propanoic acid
130: 3-(4-{[3-(3-methylbenzyloxy)benzyl]oxy}-3,5-dibromophenyl)propanoic acid
131: 3-[3,5-dibromo-4-(2-naphthylmethoxy)phenyl]-2-hydroxypropanoic acid
132: 3,5-dibromo-4-[(3-bromobenzyl)oxy]-N-(phenylsulfonyl)benzamide
133: 3-(3,5-dibromo-4-{[3-(pent-4-en-1-yloxy)benzyl]oxy}phenyl)propanoic acid
134: {3,5-dibromo-4-[2-(5,5,8,8-tetramethyl-5,6,7,8-tetrahydronaphthalen-2-yl)-2-oxoethoxy]phenyl}propanoic acid
135: 3,5-dibromo-4-[(3-bromobenzyl)oxy]benzoic acid
136: 3-[3,5-dibromo-4-({3-[(E)-2-pyridin-4-ylvinyl]benzyl}oxy)phenyl]propanoic acid
137: 3-{3,5-dibromo-4-[2-(3-methyl-1-benzothien-2-yl)-2-oxoethoxy]phenyl}propanoic acid
138: 3-{3-bromo-4-[(3-bromobenzyl)oxy]-5-piperidin-1-ylphenyl}propanoic acid
139: 3-(3,5-dibromo-4-{[3-(cyclopropylmethoxy)benzyl]oxy}phenyl)propanoic acid
140: 3-[3,5-dibromo-4-({2-[(1E)-2-methyl-3-oxobut-1-en-1-yl]benzyl}oxy)phenyl] propanoic acid
141: 3-[3,5-dibromo-4-({3-[(1E)-2-methyl-3-oxobut-1-en-1-yl]benzyloxy)phenyl] propanoic acid
142: 3,5-dibromo-O-(3-bromobenzyl)tyrosine

Especially preferred are:
11: 3-[3,5-dibromo-4-(3-methylbutoxy)phenyl]propanoic acid
13: 3-[3,5-dibromo-4-(cyclohexylmethoxy)phenyl]propanoic acid
22: 3-(3,5-dibromo-4-{[3-(trifluoromethyl)benzyl]oxy}phenyl)propanoic acid
23: 3-{3,5-dibromo-4-[(3-bromobenzyl)oxy]phenyl}propanoic acid
24: 3-{3,5-dibromo-4- [(2-methylbenzyl)oxy]phenyl } propanoic acid
25: 3-{3,5-dibromo-4-[(4-methylbenzyl)oxy]phenyl}propanoic acid
27: 3-{3,5-dibromo-4-[(4- fluorobenzyl)oxy]phenyl}propanoic acid
28: 3-(3,5-dibromo-4-{[4-(trifluoromethyl)benzyl]oxy}phenyl)propanoic acid
39: 3-{3,5-dibromo-4-[(3,5-dibromobenzyl)oxy]phenyl}propanoic acid
73: 3-(3,5-dibromo-4-{[3-fluoro-5-(trifluoromethyl)benzyl]oxy}phenyl)propanoic acid
76: 3-(4-{[3,5-bis(trifluoromethyl)benzyl]oxy}-3,5-dibromophenyl)propanoic acid
77: 3-(3,5-dibromo-4-{[3-(difluoromethoxy)benzyl]oxy}phenyl)propanoic acid
78: 3-[3,5-dibromo-4-(1-naphthylmethoxy)phenyl]propanoic acid
87: 3-{3,5-dibromo-4-[(2-fluorobenzyl)oxy]phenyl}propanoic acid
88: 3-[3,5-dibromo-4-(mesitylmethoxy)phenyl]propanoic acid
90: 3-{3,5-dibromo-4-[(3-chlorobenzyl)oxy]phenyl}propanoic acid
91: 3-{3,5-dibromo-4-[(2-chlorobenzyl)oxy]phenyl}propanoic acid
94: 3-{3,5-dibromo-4-[(2-nitrobenzyl)oxy]phenyl}propanoic acid
95: 3-(3,5-dibromo-4-{[2-(difluoromethoxy)benzyl]oxy}phenyl)propanoic acid
96: 3-(3,5-dibromo-4-{[2-(trifluoromethoxy)benzyl]oxy}phenyl)propanoic acid
98: 3-(3,5-dibromo-4-{[2-(trifluoromethyl)benzyl]oxy}phenyl)propanoic acid
99: 3-{3,5-dibromo-4-[(1-bromo-2-naphthyl)methoxy]phenyl}propanoic acid
106: 3-[3,5-dibromo-4-(3-diethylaminebenzyloxy)phenyl]propionic acid
122: 3-{3,5-dibromo-4-[2-(1H-indol-3-yl)ethoxy]phenyl}propanoic acid
123: 3-[4-(biphenyl-2-ylmethoxy)-3,5-dibromophenyl]propanoic acid
125: 2-chloro-3-{3,5-dibromo-4-[(3-bromobenzyl)oxy]phenyl}propanoic acid
126: 3-[3,5-dibromo-4-({2-[(E)-2-pyridin-4-ylvinyl]benzyl}oxy)phenyl]propanoic acid

Most preferred are:
88: 3-[3,5-dibromo-4-(mesitylmethoxy)phenyl]propanoic acid
95: 3-(3,5-dibromo-4-{[2-(difluoromethoxy)benzyl]oxy}phenyl)propanoic acid
96: 3-(3,5-dibromo-4-{[2-(trifluoromethoxy)benzyl]oxy}phenyl)propanoic acid

The following compounds represent alternatively preferred embodiments
11: 3-[3,5-dibromo-4-(3-methylbutoxy)phenyl]propanoic acid
15: 3-{3,5-dibromo-4-[(3-methylbenzyl)oxy]phenyl}propanoic acid
22: 3-(3,5-dibromo-4-{[3-(trifluoromethyl)benzyl]oxy}phenyl)propanoic acid
27: 3-{3,5-dibromo-4-[(4- fluorobenzyl)oxy]phenyl}propanoic acid
29: 3-{3,5-dibromo-4-[(3-nitrobenzyl)oxy]phenyl}propanoic acid
30: 3-{3,5-dibromo-4-[(4-tert-butylbenzyl)oxy]phenyl}propanoic acid
39: 3-{3,5-dibromo-4-[(3,5-dibromobenzyl)oxy]phenyl}propanoic acid
40: 3-{3,5-dibromo-4-[(3-cyanobenzyl)oxy]phenyl}propanoic acid
41: 3-{3,5-dibromo-4-[(3-methoxybenzyl)oxy]phenyl}propanoic acid
69: {4-[(3-bromobenzyl)oxy]-3,5-diisopropylphenyl}acetic acid
77: 3-(3,5-dibromo-4-{[3 -(difluoromethoxy)benzyl]oxy}phenyl)propanoic acid
83: 3-{3,5-dibromo-4-[(5-chloro-1-benzothien-3-yl)methoxy]phenyl}propanoic acid
88: 3-[3,5-dibromo-4-(mesitylmethoxy)phenyl]propanoic acid
95: 3-(3,5-dibromo-4-{[2-(difluoromethoxy)benzyl]oxy}phenyl)propanoic acid
100: 3-{3,5-dibromo-4-[(6-methoxy-2-naphthyl)methoxy]phenyl}propanoic acid
106: 3-[3,5-dibromo-4-(3-diethylaminebenzyloxy)phenyl]propionic acid
122: 3-{3,5-dibromo-4-[2-(1H-indol-3-yl)ethoxy]phenyl}propanoic acid
123: 3-[4-(biphenyl-2-ylmethoxy)-3,5-dibromophenyl]propanoic acid
124: 3-{3,5-dibromo-4-[(3-bromobenzyl)oxy]phenyl}-2-hydroxypropanoic acid
133: 3-(3,5-dibromo-4-{[3-(pent-4-en-1-yloxy)benzyl]oxy}phenyl)propanoic acid

Salts and solvates of compounds of formula (I) which are suitable for use in medicine are those wherein a counterion or associated solvent is pharmaceutically acceptable. However, salts and solvates having non-pharmaceutically acceptable counterions or associated solvents are within the scope of the present invention, for example, for use as intermediates in the preparation of the compounds of formula (I) and their pharmaceutically acceptable salts, solvates and physiologically functional derivatives. By the term “physiologically functional derivative” is meant a chemical derivative of a compound of formula (I) having the same physiological function as the free compound of formula (I), for example, by being convertible in the body thereto. According to the present invention, examples of physiologically functional derivatives include esters, amides, and carbamates; preferably esters and amides.

Suitable salts according to the invention include those formed with organic or inorganic acids or bases. Pharmaceutically acceptable acid addition salts include those formed from hydrochloric, hydrobromic, sulphuric, nitric, citric, tartaric, acetic, phosphoric, lactic, pyruvic, acetic, trifluoroacetic, succinic, perchloric, fumaric, maleic, glycollic, lactic, salicylic, oxaloacetic, methanesulfonic, ethanesulfonic, p-toluenesulfonic, formic, benzoic, malonic, naphthalene-2-sulfonic, benzenesulfonic, and isethionic acids. Other acids such as oxalic, while not in themselves pharmaceutically acceptable, may be useful as intermediates in obtaining the compounds of the invention and their pharmaceutical acceptable acid addition salts. Pharmaceutically acceptable base salts include ammonium salts, alkali metal salts, for example those of potassium and sodium, alkaline earth metal salts, for example those of calcium and magnesium, and salts with organic bases, for example dicyclohexylamine and N-methyl-D-glucomine.

Pharmaceutically acceptable esters and amides of the compounds of formula (I) may have an appropriate group, for example an acid group, converted to a C₁₋₆ alkyl, C₅₋₁₀ aryl, C₅₋₁₀ aryl-C₁₋₆ alkyl, or amino acid ester or amide. Pharmaceutically acceptable amides and carbonates of the compounds of formula (I) may have an appropriate group, for example an amino group, converted to a C₁₋₆ alkyl, C₅₋₁₀ aryl, C₅₋₁₀ aryl-C₁₋₆ alkyl, or amino acid ester or amide, or carbamate.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”.

A compound which, upon administration to the recipient, is capable of being converted into a compound of formula (I) as described above or an active metabolite or residue thereof, is known as a “prodrug”. A prodrug may, for example, be converted within the body, e.g. by hydrolysis in the blood, into its active form that has medical effects. Pharmaceutical acceptable prodrugs are described in T. Higuchi and V. Stella, Prodrugs as Novel Delivery Systems, Vol. 14 of the A. C. S. Symposium Series (1976); and in Edward B. Roche, ed., Bioreversible Carriers in Drug Design, American Pharmaceutical Association and Pergamon Press, 1987, both of which are incorporated herein by reference.

As used herein, the term “alky” means both straight and branched chain saturated hydrocarbon groups. Examples of alkyl groups include methyl, ethyl, n-propyl, iso-propyl, n-butyl, t-butyl, i-butyl, pentyl, hexyl, heptyl, octyl, nonyl and decyl groups. Among unbranched alkyl groups, there are preferred methyl, ethyl, n-propyl, iso-propyl, n-butyl groups. Among branched alkyl groups, there may be mentioned t-butyl, i-butyl, 1-ethylpropyl, 1-ethyl butyl and 1-ethylpentyl groups.

As used herein, the term “alkoxy” means the group O-alkyl, where “alkyl” is used as described above. Examples of alkoxy groups include methoxy and ethoxy groups. Other examples include propoxy and butoxy.

As used herein, the term “alkenyl” means both straight and branched chain unsaturated hydrocarbon groups with at least one carbon carbon double bond. Up to 5 carbon carbon double bonds may, for example, be present. Examples of alkenyl groups include ethenyl, propenyl, butenyl, pentenyl, hexenyl, heptenyl, octenyl, nonenyl, decenyl and dodecenyl. Preferred alkenyl groups includes ethenyl, 1-propenyl and 2-propenyl.

As used herein, the term “alkenyloxy” means the group O-alkenyl, where “alkenyl” is used as described above. Examples of alkenyloxy groups include ethenyloxy groups. Other examples include 2-propenyloxy, 3-butenyloxy and 4-pentenyloxy.

As used herein, the term “alkynyl” means both straight and branched chain unsaturated hydrocarbon groups with at least one carbon carbon triple bond. Up to 5 carbon carbon triple bonds may, for example, be present. Examples of alkynyl groups include ethynyl, propynyl, butynyl, pentynyl, hexynyl, heptynyl, octynyl, nonynyl, decynyl and dodecynyl. Preferred alkenyl groups include ethynyl 1-propynyl and 2-propynyl.

As used herein, the term “cycloalkyl” means a saturated group in a ring system. The cycloalkyl group can be monocyclic or bicyclic. A bicyclic group may, for example, be fused or bridged. Examples of monocyclic cycloalkyl groups include cyclopropyl, cyclobutyl and cyclopentyl. Other examples of monocyclic cycloalkyl groups are cyclohexyl, cycloheptyl and cyclooctyl. Examples of bicyclic cycloalkyl groups include bicyclo [2.2.1]hept-2-yl. Preferably, the cycloalkyl group is monocyclic.

As used herein, the term “cycloalkenyl” means an unsaturated aliphatic group in a ring system. A cycloalkenyl group can be monocyclic or bicyclic. Preferably, the cycloalkyl group is monocyclic. Examples of monocyclic cycloalkenyl groups include cyclopentenyl and cyclohexenyl.

As used herein, the term “aryl” means a monocyclic or bicyclic aromatic carbocyclic group. Examples of aryl groups include phenyl and naphthyl. A naphthyl group may be attached through the 1 or the 2 position. In a bicyclic aromatic group, one of the rings may, for example, be partially saturated. Examples of such groups include indanyl and tetrahydronaphthyl. Specifically, the term C₅₋₁₀ aryl is used herein to mean a group comprising from 5 to 10 carbon atoms in a monocyclic or bicyclic aromatic group. A particularly preferred C₅₋₁₀ aryl group is phenyl.

As used herein, the term “aryloxy” means the group O-aryl, where “aryl” is used as described above.

As used herein, the term “halogen” means fluorine, chlorine, bromine or iodine. Fluorine, chlorine and bromine are particularly preferred.

As used herein, the term “heterocyclyl” means an aromatic (“heteroaryl”) or a non-aromatic (“heterocycloalkyl”) cyclic group of carbon atoms wherein from one to three of the carbon atoms is/are replaced by heteroatoms independently selected from nitrogen, oxygen and sulfur. A heterocyclyl group may, for example, be monocyclic or bicyclic. In a bicyclic heterocyclyl group there may be one or more heteroatoms in each ring, or only in one of the rings. The heteroatom is preferably O or N. Heterocyclyl groups containing a suitable nitrogen atom include the corresponding N-oxides.

Examples of monocyclic heterocycloalkyl rings include aziridinyl, azetidinyl, pyrrolidinyl, imidazolidinyl, pyrazolidinyl, piperidinyl, piperazinyl, tetrahydrofuranyl, tetrahydropyranyl, morpholinyl, thiomorpholinyl and azepanyl.

Examples of bicyclic heterocyclic rings in which one of the rings is non-aromatic include dihydrobenzofuranyl, indanyl, indolinyl, isoindolinyl, tetrahydroisoquinolinyl, tetrahydroquinolyl and benzoazepanyl.

Examples of monocyclic heteroaryl groups include furanyl, thienyl, pyrrolyl, oxazolyl, thiazolyl, imidazolyl, oxadiazolyl, thiadiazolyl, pyridyl, triazolyl, triazinyl, pyridazyl, pyrimidinyl, isothiazolyl, isoxazolyl, pyrazinyl, pyrazolyl and pyrimidinyl; examples of bicyclic heteroaryl groups include quinoxalinyl, quinazolinyl, pyridopyrazinyl, benzoxazolyl, benzothiophenyl, benzimidazolyl, naphthyridinyl, quinolinyl, benzofuranyl, indolyl, benzothiazolyl, oxazolyl[4,5-b]pyridiyl, pyridopyrimidinyl, isoquinolinyl and benzodroxazole.

Examples of preferred heterocyclyl groups include piperidinyl, tetrahydrofuranyl, tetrahydropyranyl, pyridyl, pyrimidyl and indolyl.

As used herein, the term “arylalkyl” means a group aryl-alkyl- attached through the alkyl group, “aryl” and “alkyl” being understood to have the meanings outlined above.

As used herein the term “cycloalkylalkyl” means a group cycloalkyl-alkyl- attached through the alkyl group, “cycloalkyl” and “alkyl” being understood to have the meanings outlined above.

As used herein the term “cycloalkylalkoxy” means a group cycloalkyl-alkoxy- attached through the alkoxy group, “cycloalkyl” and “alkoxy” being understood to have the meanings outlined above.

As used herein the term “arylalkoxy” means a group aryl-alkoxy- attached through the alkoxy group, “aryl” and “alkoxy” being understood to have the meanings outlined above.

As used herein, the term “heterocyclylalkyl” means a group heterocyclyl-alkyl- attached through the alkyl group, “heterocyclyl” and “alkyl” being understood to have the meanings outlined above. Similarly, as used herein, the term “heterocyclylalkenyl” means a group heterocyclyl-alkenyl- attached through the alkenyl group, “heterocyclyl” and “alkenyl” being understood to have the meanings outlined above.

As mentioned above, the compounds of the invention have activity as antagonists, partial antagonists or partial agonists to the action of endogenous hormones, for example testosterone or dihydrotestosterone, at the androgen receptor. Therefore, the compounds that are antagonists or partial antagonists have use in the treatment or prophylaxis of clinical conditions for which an antagonist or a partial antagonist of the androgen receptor is indicated. Such conditions include cancers, bone diseases, reproductive diseases and others. In particular, there may be mentioned prostate cancer, psychological abnormalities including, but not limited to mood (for example aggression and anxiety), male pattern baldness (alopecia), benign prostatic hyperplasia (BPH), amenorrhea, hypogonadism, anemia, defects in spermatogenesis, cachexia, osteoporosis, osteopenia, and muscle wasting. Compounds that are partial agonists to the action of the endogenous hormones have use in the or prophylaxis of clinical conditions for which a partial agonist of the androgen receptor is indicated. Such conditions include cardiovascular disease and psychological abnormalities, including mood (for example depression) and cognitive function.

The compounds of the invention find particular application in the treatment or prophylaxis of prostate cancer.

Accordingly, the invention also provides a method for the treatment or prophylaxis of a condition in a mammal mediated by an androgen receptor, which comprises administering to the mammal a therapeutically effective amount of a compound of formula (I) as defined above or a pharmaceutically acceptable ester, amide, solvate or salt thereof, including a salt of such an ester or amide, and a solvate of such an ester, amide or salt.

The invention also provides the use of a compound of formula (I) as defined above or a pharmaceutically acceptable ester, amide, solvate or salt thereof, including a salt of such an ester or amide, and a solvate of such an ester, amide or salt, for the manufacture of a medicament for the treatment or prophylaxis of a condition mediated by an androgen receptor.

Hereinafter, the term “active ingredient” means a compound of formula (I) as defined above, or a pharmaceutically acceptable ester, amide, solvate or salt thereof, including a salt of such an ester or amide, and a solvate of such an ester, amide or salt.

The amount of active ingredient which is required to achieve a therapeutic effect will, of course, vary with the particular compound, the route of administration, the subject under treatment, and the particular disorder or disease being treated. The compounds of the invention may be administered orally or via injection at a dose of from 0.1 to 1500 mg/kg per day, preferably 0.1 to 500 mg/kg per day. The dose range for adult humans is generally from 5 mg to 35 g per day and preferably 5 mg to 2 g per day. Tablets or other forms of presentation provided in discrete units may conveniently contain an amount of compound of the invention which is effective at such dosage or as a multiple of the same, for example units containing 5 mg to 500 mg, usually around 10 mg to 200 mg.

While it is possible for the active ingredient to be administered alone, it is preferable for it to be present in a pharmaceutical formulation. Accordingly, the invention provides a pharmaceutical formulation comprising a compound of formula (I) as defined above or a pharmaceutically acceptable ester, amide, solvate or salt thereof, including a salt of such an ester or amide, and a solvate of such an ester, amide or salt, and a pharmaceutically acceptable excipient.

The pharmaceutical formulations according to the invention include those suitable for oral, parenteral (including subcutaneous, intradermal, intramuscular, intravenous, and intraarticular), inhalation (including fine particle dusts or mists which may be generated by means of various types of metered does pressurized aerosols), nebulizers or insufflators, rectal and topical (including dermal, buccal, sublingual, and intraocular) administration, although the most suitable route may depend upon, for example, the condition and disorder of the recipient.

The formulations may conveniently be presented in unit dosage form and may be prepared by any of the methods well known in the art of pharmacy. All methods include the step of bringing the active ingredient into association with the carrier which constitutes one or more accessory ingredients. In general the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein.

Formulations for parenteral administration include aqueous and non-aqueous sterile injection solutions which may contain anit-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example saline or water-for-injection, immediately prior to use. Extemporaneous injection solutions and suspensions may be prepared from sterile powders, granules and tablets of the kind previously described.

Formulations for rectal administration may be presented as a suppository with the usual carriers such as cocoa butter or polyethylene glycol.

Formulations for topical administration in the mouth, for example buccally or sublingually, include lozenges comprising the active ingredient in a flavoured basis such as sucrose and acacia or tragacanth, and pastilles comprising the active ingredient in a basis such as gelatin and glycerine or sucrose and acacia.

Preferred unit dosage formulations are those containing an effective dose, as hereinbefore recited, or an appropriate fraction thereof, of the active ingredient.

It should be understood that in addition to the ingredients particularly mentioned above, the formulations of this invention may include other agents conventional in the art having regard to the type of formulation in question, for example those suitable for oral administration may include flavouring agents.

The compounds of formula (I) as described above also find use, optionally in labelled form, as a diagnostic agent for the diagnosis of conditions associated with malfunction of the androgen receptor. For example, such a compound may be radioactively labelled.

The compounds of formula (I) as described above also find use as a reference compound in methods of discovering other antagonists partial antagonists or partial agonists of the androgen receptor. Thus, the invention provides a method of discovering a ligand of the androgen receptor which comprising use of a compound of the invention or a compound of the invention in labelled form, as a reference compound. For example, such a method may involve a competitive binding experiment in which binding of a compound of formula (I) to the androgen receptor is reduced by the presence of a further compound which has androgen receptor-binding characteristics, for example stronger androgen receptor-binding characteristics than the compound of formula (I) in question.

In a further aspect, the invention provides a compound of formula (Ib) or a pharmaceutically acceptable ester, amide, solvate or salt thereof, including a salt of such an ester or amide, and a solvate of such an ester, amide or salt,

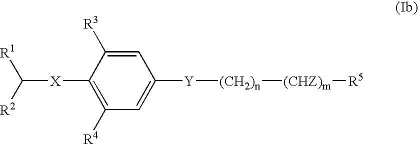

wherein:
R¹ is selected from C₅₋₁₀ aryl, C(O)—C₅₋₁₀ aryl, C(O)—C₃₋₈ heterocyclyl, C₅₋₁₀ aryl-C₁₋₂ alkyl, C₃₋₁₀ heterocyclyl, C₃₋₁₀ heterocyclyl-C₁₋₂ alkyl, C₃₋₁₅ alkyl, C₄₋₁₅ alkenyl, C₃₋₁₅ alkynyl, C₃₋₁₀ cycloalkyl and C₃₋₁₀ cycloalkyl C₁₋₂ alkyl, said alkyl, alkenyl and alkynyl optionally being substituted with, where applicable, 1 to 3 groups R^{a} which may be the same or different; said aryl-alkyl, heterocyclyl and cycloalkyl optionally being substituted with, where applicable, 1 to 3 groups R^{a′} which may be the same or different; said aryl optionally being substituted with, where applicable, 1 to 4 groups R^{a″} which may be the same or different;

### R² is selected from hydrogen, C₁₋₄ alkyl, C₂₋₄ alkenyl, C₂₋₄ alkynyl and C₁₋₄ alkoxy;

or R¹ and R² together with the carbon atom to which they are both attached form a C₄₋₈ cycloalkyl, C₄₋₈ cycloalkenyl, a saturated or partially saturated C₃₋₁₀ heterocyclyl, optionally substituted with, where applicable, 1 to 3 groups R^{a′} which may be the same or different;

### X is selected from CH₂, oxygen, sulfur, sulfoxide, sulfone, selenium, tellurium, disulfide, and a group of formula —N(R^{c})—;

R³ and R⁴ are independently selected from hydrogen, halogen, C₁₋₄ alkyl, C₃₋₇ cycloalkyl, C₃₋₇ heterocyclyl, C₂₋₄ alkenyl, C₂₋₄ alkynyl, C₁₋₄ alkoxy, fluoromethyl, difluoromethyl, trifluoromethyl, fluoromethoxy, difluoromethoxy, trifluoromethoxy, methylthio, fluoromethylthio, difluoromethylthio, trifluoromethylthio, and COOR^{c};

### Y is selected from bond, carbonyl, oxygen, sulphur, —CH(R^{b})—, —NHCO—, —NHSO₂—, —SO₂NH—, —NR^{c})— and —CR⁶═CR⁷—;

n is selected from 0, 1, 2 and 3;

### Z is selected from halogen, amino, hydroxy, mercapto, C₁₋₄ alkyl, C₂₋₄ alkenyl, C₂₋₄ alkynyl, C₁₋₄ alkoxy, fluoromethyl, difluoromethyl, trifluoromethyl, fluoromethoxy, difluoromethoxy, trifluoromethoxy and (CH₂)ₚOH, where p is an integer from 1 to 4;

### R⁵ is selected from —CO₂R^{c}, —PO(OR^{c})₂, —PO(OR^{c})NH_{2,} —SO₂OR^{c}, —COCO₂R^{c}, CONR^{c}OR^{c}, —SO₂NHR^{c}, —NHSO₂R^{c′}, —CONHSO₂R^{c}, and —SO₂NHCOR^{c};

R⁶ and R⁷ are independently selected from hydrogen, halogen, C₁₋₄ alkyl, C₂₋₄ alkenyl, C₂₋₄ alkynyl, C₁₋₄ alkoxy, C₅₋₁₀ aryl, fluoromethyl, difluoromethyl, trifluoromethyl, fluoromethoxy, difluoromethoxy, trifluoromethoxy, and (CH₂)ₚOH, where p is an integer from 1 to 4;

### R^{a} is selected from halogen, C₁₋₄ alkoxy, C₅₋₁₀ aryl, fluoromethoxy, difluoromethoxy, trifluoromethoxy, methylthio, fluoromethylthio, difluoromethylthio, trifluoromethylthio, mercapto, cyano, and nitro;

### R^{a′} is selected from R^{a}, fluoromethyl, difluoromethyl, trifluoromethyl, C₁₋₄ alkyl, C₃₋₁₀ heterocyclyl-C₂₋₄ alkenyl, C₅₋₁₀ aryl-C₂₋₄ alkenyl, C₃₋₁₀ heterocyclyl-C₁₋₄ alkyl and C₅₋₁₀ aryl-C₁₋₄ alkyl;

R^{a″} is selected from:
R^{a′};
C₂₋₄ alkenyl, optionally substituted with 1, 2 or 3 groups selected from C₅₋₁₀ aryl, C(O)R^{c}, C₃₋₁₀ heterocyclyl, and C₃₋₁₀ heterocyclyl substituted with C₁₋₄ alkyl;
C₂₋₈ alkenyloxy;
C₃₋₈ cycloalkyl-C₁₋₃ alkoxy, C₅₋₁₀ aryl-C₁₋₃ alkoxy, or C₅₋₁₀ aryloxy, said C₃₋₈ cycloalkyl-C₁₋₃ alkoxy, C₅₋₁₀ aryl-C₁₋₃ alkoxy or C₅₋₁₀ aryloxy optionally being substituted with 1, 2 or 3 groups selected from C₁₋₄ alkyl, halogen, fluoromethyl, difluoromethyl, trifluoromethyl, fluoromethoxy, difluoromethoxy, trifluoromethoxy, methylthio, mercapto, hydroxy, cyano, nitro, a group of formula —NR^{c})₂ in which the two R^{c} groups may be the same or different but not both simultaneously hydrogen;
   R^{b} is selected from hydrogen, halogen, hydroxyl, mercapto, C₁₋₄ alkyl, C₂₋₄ alkenyl, C₂₋₄ alkynyl, C₁₋₄ alkoxy, fluoromethyl, difluoromethyl, trifluoromethyl, fluoromethoxy, difluoromethoxy, trifluoromethoxy and (CH₂)ₚOH, where p is an integer from 1 to 4; and

### R^{c′} is selected from hydrogen, C₁₋₄ alkyl, C₂₋₄ alkenyl and C₂₋₄ alkynyl;

### R^{c′} is selected from R^{c}, C₅₋₁₀ aryl or C₅₋₁₀ aryl substituted with amino, hydroxyl, halogen or C₁₋₄ alkyl; and

m is 1; or
simultaneously m is 0 or 1 and R³ is C₃₋₇ heterocyclyl; or
simultaneously Y is bond, m is 0, n is 0 and R⁵ is —CO₂R^{c}.

The invention further provides a compound of formula (Ic) or a pharmaceutically acceptable ester, amide, solvate or salt thereof A compound of formula (Ic) or a pharmaceutically acceptable ester, amide, solvate or salt thereof, including a salt of such an ester or amide, and a solvate of such an ester, amide or salt,

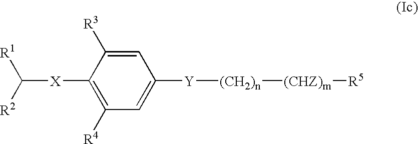

wherein:

### R¹ is selected from C₅₋₁₀ aryl, C(O)—C₅₋₁₀ aryl, C(O)—C₃₋₈ heterocyclyl or C₅₋₁₀ heterocyclyl-C₁₋₂ alkyl,

said C(O)—C₅₋₁₀ aryl, C(O)—C₃₋₈ heterocyclyl or C₅₋₁₀ heterocyclyl-C₁₋₂ alkyl optionally being substituted with, where applicable, 1 to 3 groups R^{a′} which may be the same or different;
said C₅₋₁₀ aryl being substituted with a group selected from:
   C₅₋₁₀ aryl, fluoromethoxy, difluoromethoxy, trifluoromethoxy, mercapto, fluoromethyl, difluoromethyl, and C₃₋₁₀ heterocyclyl-C₂₋₄ alkenyl;
   C₂₋₄ alkenyl, substituted with 1, 2 or 3 groups selected from C₅₋₁₀ aryl, C(O)R^{c}, C₃₋₁₀ heterocyclyl, and C₃₋₁₀ heterocyclyl substituted with C₁₋₄ alkyl;
   C₂₋₈ alkenyloxy;
   C₃₋₈ cycloalkyl-C₁₋₃ alkoxy, C₅₋₁₀ aryl-C₁₋₃ alkoxy, or C₅₋₁₀ aryloxy, said C₃₋₈ cycloalkyl-C₁₋₃ alkoxy, C₅₋₁₀ aryl-C₁₋₃ alkoxy or C₅₋₁₀ aryloxy optionally being substituted with 1, 2 or 3 groups selected from C₁₋₄ alkyl, halogen, fluoromethyl, difluoromethyl, trifluoromethyl, fluoromethoxy, difluoromethoxy, trifluoromethoxy, methylthio, mercapto, hydroxy, cyano, nitro, a group of formula —N(R^{c})₂ in which the two R^{c} groups may be the same or different but not both simultaneously hydrogen;
said aryl optionally also substituted with, where applicable, 1 to 2 groups R^{a′} which may be the same or different,

### R² is selected from hydrogen, C₁₋₄ alkyl, C₂₋₄ alkenyl, C₂₋₄ alkynyl and C₁₋₄ alkoxy;

### X is selected from CH₂, oxygen, sulfur, sulfoxide, sulfone, selenium, tellurium, disulfide, and a group of formula —N(R^{c})—;

R³ and R⁴ are independently selected from hydrogen, halogen, C₁₋₄ alkyl, C₃₋₇ cycloalkyl, C₃₋₇ heterocyclyl, C₂₋₄ alkenyl, C₂₋₄ alkynyl, C₁₋₄ alkoxy, fluoromethyl, difluoromethyl, trifluoromethyl, fluoromethoxy, difluoromethoxy, trifluoromethoxy, methylthio, fluoromethylthio, difluoromethylthio, trifluoromethylthio, and COOR^{c};

### Y is selected from bond, carbonyl, oxygen, sulphur, —CH(R^{b})—, —NHCO—, —NHSO₂—, —SO₂NH—, —N(R^{c})— and —CR⁶═CR7—;

n is selected from 0, 1, 2 and 3;

### Z is selected from halogen, amino, hydroxy, mercapto, C₁₋₄ alkyl, C₂₋₄ alkenyl, C₂₋₄ alkynyl, C₁₋₄ alkoxy, fluoromethyl, difluoromethyl, trifluoromethyl, fluoromethoxy, difluoromethoxy, trifluoromethoxy and (CH₂)ₚOH, where p is an integer from 1 to 4;

m is selected from 0 and 1;

### R⁵ is selected from —CO₂R^{c}, —PO(OR^{c})₂, —PO(OR^{c})NH₂, —SO₂OR^{c}, —COCO₂R^{c}, CONR^{c}OR^{c}, —SO₂NHR^{c}, —NHSO₂R^{c′}, —CONHSO₂R^{c}, and —SO₂NHCOR^{c};

R⁶ and R⁷ are independently selected from hydrogen, halogen, C₁₋₄ alkyl, C₂₋₄ alkenyl, C₂₋₄ alkynyl, C₁₋₄ alkoxy, C₅₋₁₀ aryl, fluoromethyl, difluoromethyl, trifluoromethyl, fluoromethoxy, difluoromethoxy, trifluoromethoxy, and (CH₂)ₚOH, where p is an integer from 1 to 4;

### R^{a} is selected from halogen, C₁₋₄ alkoxy, C₅₋₁₀ aryl, fluoromethoxy, difluoromethoxy, trifluoromethoxy, methylthio, fluoromethylthio, difluoromethylthio, trifluoromethylthio, mercapto, cyano, and nitro;

### R^{a′} is selected from R^{a}, fluoromethyl, difluoromethyl, trifluoromethyl, C₁₋₄ alkyl, and C₃₋₁₀ heterocyclyl-C₂₋₄ alkenyl;

R^{b} is selected from hydrogen, halogen, hydroxyl, mercapto, C₁₋₄ alkyl, C₂₋₄ alkenyl, C₂₋₄ alkynyl, C₁₋₄ alkoxy, fluoromethyl, difluoromethyl, trifluoromethyl, fluoromethoxy, difluoromethoxy, trifluoromethoxy and (CH₂)ₚOH, where p is an integer from 1 to 4; and

### R^{c} is selected from hydrogen, C₁₋₄ alkyl, C₂₋₄ alkenyl and C₂₋₄ alkynyl; and

### R^{c′} is selected from R^{c}, C₅₋₁₀ aryl or C₅₋₁₀ aryl substituted with amino, hydroxyl, halogen or C₁₋₄ alkyl.

It will be understood that the preferred features mentioned above in respect of compounds of formula (I) for use in the invention, and the uses of those compounds also apply to compounds of formula (Ib) and (Ic).

Accordingly, a preferred group of compounds according to formula (Ib) includes compounds according to formula (Ib′) or a pharmaceutically acceptable ester, amide, solvate or salt thereof, including a salt of such an ester or amide, and a solvate of such an ester, amide or salt

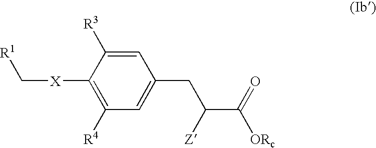

wherein:
R¹ is selected from C₆₋₁₀ aryl, C₅₋₁₀ heterocyclyl-C₁₋₂ alkyl, C₄₋₁₀ alkyl and C₅₋₇ cycloalkyl, said alkyl optionally being substituted with, where applicable, 1 to 3 groups R^{a} which may be the same or different; said cycloalkyl optionally being substituted with, where applicable, 1 to 3 groups R^{a′} which may be the same or different; said aryl optionally being substituted with, where applicable, 1 to 4 groups R^{a″} which may be the same or different;

### X is selected from oxygen and sulfur;

### R³ and R⁴ are independently selected from hydrogen, halogen, C₁₋₂ alkyl, C₁₋₂ alkoxy, fluoromethyl, difluoromethyl, trifluoromethyl, fluoromethoxy, difluoromethoxy and trifluoromethoxy;

### Z′ is selected from halogen, hydroxy and mercapto;

### R^{a} is selected from halogen, C₅₋₁₀ aryl, fluoromethoxy, difluoromethoxy, trifluoromethoxy and nitro;

### R^{a′} is selected from R^{a}, fluoromethyl, difluoromethyl, trifluoromethyl, C₁₋₄ alkyl, and C₅₋₁₀ heterocyclyl-C₂₋₄ alkenyl, C₅₋₁₀ aryl-C₂₋₄ alkenyl, C₅₋₁₀ heterocyclyl-C₁₋₄ alkyl and C₅₋₁₀ aryl-C₁₋₄ alkyl;

R^{a″} is selected from:

R^{a′};
C₂₋₄ alkenyl, substituted with C₃₋₁₀ heterocyclyl;
C₅₋₁₀ aryloxy, optionally being substituted with 1, 2 or 3 groups selected from C₁₋₄ alkyl, halogen, fluoromethyl, difluoromethyl, trifluoromethyl, fluoromethoxy, difluoromethoxy, trifluoromethoxy, methylthio, mercapto, hydroxy, cyano or nitro; and

### R^{c} is selected from hydrogen and C₁₋₄ alkyl.

Further, a preferred group of compounds according to formula (Ic) includes compounds according to formula (Ic′) or a pharmaceutically acceptable ester, amide, solvate or salt thereof, including a salt of such an ester or amide, and a solvate of such an ester, amide or salt

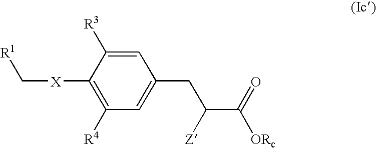

### R¹ is selected from C₆₋₁₀ aryl or C₅₋₁₀ heterocyclyl-C₁₋₂ alkyl,

said C₅₋₁₀ heterocyclyl-C₁₋₂ alkyl optionally being substituted with, where applicable, 1 to 3 groups R^{a′} which may be the same or different;
said C₆₋₁₀ aryl being substituted with a group selected from:
   C₅₋₁₀ aryl, fluoromethoxy, difluoromethoxy, trifluoromethoxy, mercapto, fluoromethyl, difluoromethyl, and C₃₋₁₀ heterocyclyl-C₂₋₄ alkenyl;
   C₂₋₄ alkenyl, substituted with 1, 2 or 3 groups selected from C₅₋₁₀ aryl, C(O)R^{c}, C₃₋₁₀ heterocyclyl, and C₃₋₁₀ heterocyclyl substituted with C₁₋₄ alkyl;
   C₂₋₈ alkenyloxy;
   C₃₋₈ cycloalkyl-C₁₋₃ alkoxy, C₅₋₁₀ aryl-C₁₋₃ alkoxy, or C₅₋₁₀ aryloxy, said C₃₋₈ cycloalkyl-C₁₋₃ alkoxy, C₅₋₁₀ aryl-C₁₋₃ alkoxy or C₅₋₁₀ aryloxy optionally being substituted with 1, 2 or 3 groups selected from C₁₋₄ alkyl, halogen, fluoromethyl, difluoromethyl, trifluoromethyl, fluoromethoxy, difluoromethoxy, trifluoromethoxy, methylthio, mercapto, hydroxy, cyano, nitro, a group of formula —NR^{c})₂ in which the two R^{c} groups may be the same or different but not both simultaneously hydrogen;
said aryl optionally also substituted with, where applicable, 1 to 2 groups R^{a′} which may be the same or different,

### X is selected from oxygen and sulfur;

### R³ and R⁴ are independently selected from hydrogen, halogen, C₁₋₂ alkyl, C₁₋₂ alkoxy, fluoromethyl, difluoromethyl, trifluoromethyl, fluoromethoxy, difluoromethoxy and trifluoromethoxy;

### Z′ is selected from hydrogen, halogen, hydroxyl and mercapto;

### R^{a} is selected from halogen, C₅₋₁₀ aryl, fluoromethoxy, difluoromethoxy, trifluoromethoxy and nitro;

### R^{a′} is selected from R^{a}, fluoromethyl, difluoromethyl, trifluoromethyl, C₁₋₄ alkyl, and C₅₋₁₀ heterocyclyl-C₂₋₄ alkenyl; and

### R^{c} is selected from hydrogen and C₁₋₄ alkyl.

The invention also provides a method for preparing a compound of formula (Ib) as described above or a compound of formula (Ic) as described above comprising a step of adding a compound of formula (II)

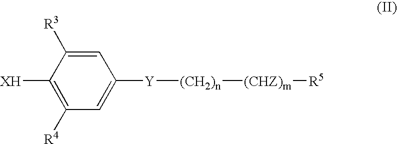

wherein X, R³, R⁴, Y, n, Z, m and R⁵ are as defined above for the compound of formula (Ib) or (Ic), to a compound of formula (III)

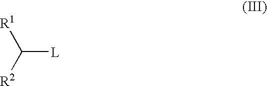

wherein R¹ and R² are as defined above, as appropriate, for the compound of formula (Ib) or (Ic) and L is a suitable leaving group, in the presence of a suitable base.

Suitable leaving groups L include halogen, OR^{c}, —SR^{c}, C₁₋₄alkyl, C₅₋₁₀ aryl or C₅₋₁₀ aryl-C₁₋₄ alkyl sulphonate esters, for example, a bromide, a methylsulfonyl or a toluenesufonyl group. Suitable bases include carbonates, alkylamines and alkali metal hydroxides, for example potassium carbonate, cesium carbonate, potassium hydroxide, sodium hydroxide diisopropylamine and, triethylamine. Trimethylsilanoate may also be used. Other combinations of leaving groups and bases may be employed, as is known by the person skilled in the art. Optionally, one or more coupling reagents may be employed. The reaction mixture is stirred at room temperature, or heated until the starting materials have been consumed. The reaction may be carried out with protecting groups present and those protecting groups may be removed after the reaction. Suitable protecting groups are known to the person skilled in the art (see T. W. Greene, “Protective Groups in Organic Synthesis”, 3rd Edition, New York, 1999).

The invention will now be illustrated by the following Examples, which do not in any way limit the scope of the invention.

### EXAMPLES

The following compounds illustrate compounds of the invention or, where appropriate, compounds for use in the invention.

Example compounds 1 to 30 are shown in Table 1.

TABLE 1

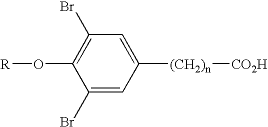

| Ex- | | | | | |
|---|---|---|---|---|---|
| am- | | | | | |
| ple | R | n | Method | Yield¹ | MS² |
| 1 | 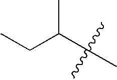 | 1 | A | 89 | 365.2 |
| 2 | 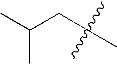 | 1 | A | 32 | 365.2 |
| 3 | 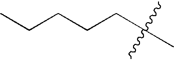 | 1 | A | 81 | 379 |
| 4 | 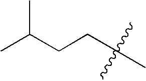 | 1 | A | 90 | 379 |
| 5 | 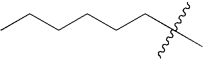 | 1 | A | 71 | 393.1 |
| 6 | 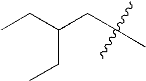 | 1 | A | 79 | 393.1 |
| 7 | 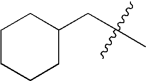 | 1 | A | 74 | 405.1 |
| 8 | 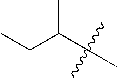 | 2 | A | 68 | 379 |
| 9 | 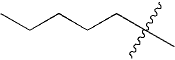 | 2 | A | 88 | 293.1 |
| 10 | 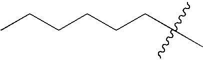 | 2 | A | 92 | 407.2 |
| 11 | 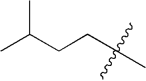 | 2 | A | 90 | 393.1 |
| 12 | 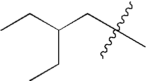 | 2 | A | 70³ | 407.2 |
| 13 | 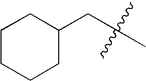 | 2 | A | 63 | 419.2 |
| 14 | 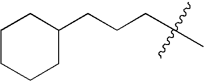 | 2 | B | 30 | — |
| 15 | 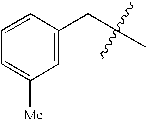 | 2 | B | 66 | 428.1 |
| 16 | 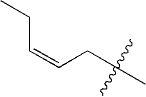 | 2 | B | 64 | 391 |
| 17 | 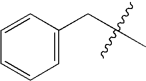 | 2 | B | 82 | 412.9 |
| 18 | 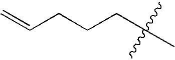 | 2 | B | 77 | 390.4 |
| 19 | 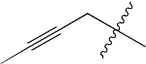 | 2 | B | 50 | — |
| 20 | 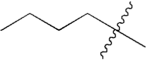 | 1 | A | 96 | 365.2 |
| 21 | 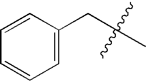 | 1 | C | 37 | 400.1 |
| 22 | 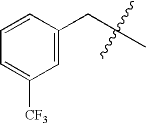 | 2 | C | 84 | 482.1 |
| 23 | 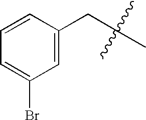 | 2 | C | 67 | 493 |
| 24 | 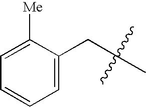 | 2 | C | 97 | 428.1 |
| 25 | 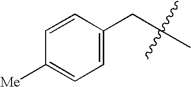 | 2 | C | 95 | 428.1 |
| 26 | 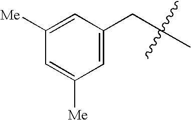 | 2 | C | 75 | 442.1 |
| 27 | 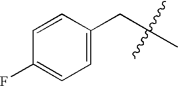 | 2 | C⁴ | 5 | 432.1 |
| 28 | 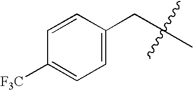 | 2 | C⁴ | 42 | 482.1 |
| 29 | 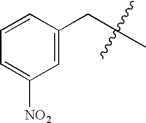 | 2 | C | 65 | 459.1 |
| 30 | 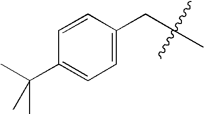 | 2 | C⁴ | 4 | 470.2 |

¹Yields in %.
²MS result obtained on a Perkin-Elmer API 150Ex spectrometer, using electrospray negative ion mode.
³This substance fell as an oil during final work-up. In order to get crystals the methanol was removed in vacuo and the remaining water phase, extracted with ethyl acetate. After drying over magnesium sulphate and removal of the organic phase, a crystal mass was obtained.
⁴In Examples 27, 28 and 30, the final compound was first separated in 2 g silica pre-packed in 3 mL SPE cartridges employing the same gradient of solvents described in Procedure C and then further purified by semi-preparative-HPLC (Zorbax CombiHT (SB-C8 50 × 21.2 mm, 5μ) Mobil Phase: Solvent A. Water with 0.5% formic acid; Solvent B: acetonitrile. Gradient: 2 min 80% of A then over 8 min to 5% of A).

Example compounds 31 to 35 are the following:
31: N-[3,5-Dibromo-4-(3-bromobenzyloxy)benzoyl]benzenesulphonamide
32: N-[3,5-Dibromo-4-(3-bromobenzyloxy)benzoyl]-3-nitrobenzenesulphonamide
33: N-[3,5-Dibromo-4-(3-bromobenzyloxy)benzoyl]-4-nitrobenzenesulphonamide
34: 4-Amino-N-[3,5-dibromo-4-(3-bromobenzyloxy)benzoyl]benzenesulphonamide
35: N-[3,5-Dibromo-4-(3-bromobenzyloxy)benzoyl]methanesulphonamide

Example compounds 36 to 100 are shown in Table 2:

TABLE 2

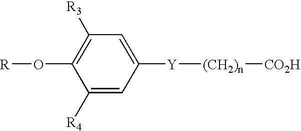

| | | | | | | Equiv. | Hydrol. | Yield | |
|---|---|---|---|---|---|---|---|---|---|
| Example | R | R₃, R₄ | Y | n | Solvent | halide | Method | (%) | MS¹ |
| 36 | 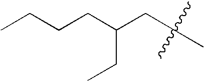 | Br | CH₂ | 0 | acetone | 4 | D1 | 27 | 421.0 |
| 37 | 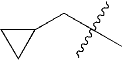 | Br | CH₂ | 0 | acetone | 4 | D4 | 75 | 363.1 |
| 38 | 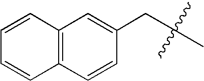 | Br | CH₂ | 1 | acetone | 4 | D2 | 34 | 463.0 |
| 39 | 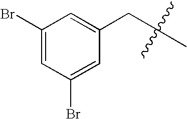 | Br | CH₂ | 1 | CH₃CN | 2 | D3 | 38 | 570.7 |
| 40 | 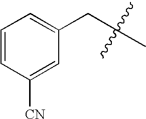 | Br | CH₂ | 1 | CH₃CN | 2 | D3 | 51 | 437.8 |
| 41 | 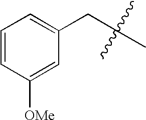 | Br | CH₂ | 1 | CH₃CN | 2 | D3 | 91 | 443.2 |
| 42 | 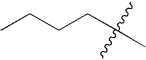 | Cl | CHOH | 0 | CH₃CN | 2 | D4 | 58 | 290.8 |
| 43 | 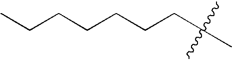 | Cl | CHOH | 0 | CH₃CN | 2 | D4 | 52 | 333.1 |
| 44 | 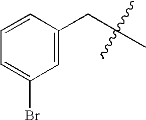 | Cl | CHOH | 0 | CH₃CN | 2 | D4 | 64 | 404.8 |
| 45 | 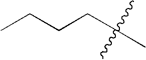 | Cl | C═O | 0 | CH₃CN | 2 | D4 | 48 | 289.0 |
| 46 | 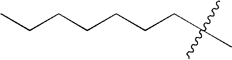 | Cl | C═O | 0 | CH₃CN | 2 | D4 | 12 | 330.7 |
| 47 | 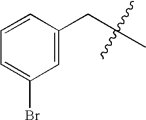 | Cl | C═O | 0 | CH₃CN | 2 | D4 | 64 | 402.7 |
| 48 | 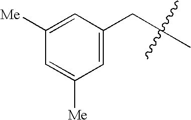 | Cl | C═O | 0 | CH₃CN | 2 | D4 | 7 | 351.1 |
| 49 | 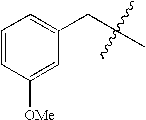 | Cl | C═O | 0 | CH₃CN | 2 | D4 | 63 | 352.9 |
| 50 | 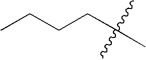 | Me | CH₂ | 0 | CH₃CN | 2 | D4 | 61 | 235.0 |
| 51 | 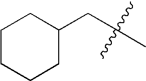 | Me | CH₂ | 0 | CH₃CN | 2 | D4 | 46 | 275.2 |
| 52 | 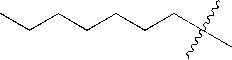 | Me | CH₂ | 0 | CH₃CN | 2 | D4 | 61 | 277.0 |
| 53 | 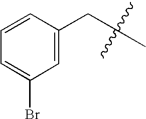 | Me | CH₂ | 0 | CH₃CN | 2 | D4 | 78 | 349.0 |
| 54 | 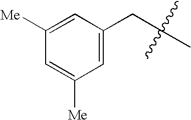 | Me | CH₂ | 0 | CH₃CN | 2 | D4 | 76 | 297.1 |
| 55 | 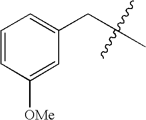 | Me | CH₂ | 0 | CH₃CN | 2 | D4 | 85 | 299.2 |
| 56 | 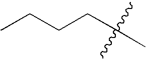 | Me | C═O | 0 | CH₃CN | 2 | D4 | 43 | 248.8 |
| 57 | 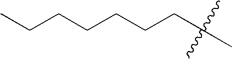 | Me | C═O | 0 | CH₃CN | 2 | D4 | 82 | 290.8 |
| 58 | 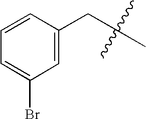 | Me | C═O | 0 | CH₃CN | 2 | D4 | 93 | 361.0 |
| 59 | 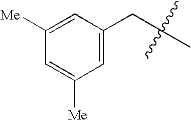 | Me | C═O | 0 | CH₃CN | 2 | D4 | 85 | 310.9 |
| 60 | 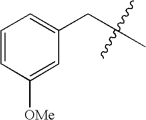 | Me | C═O | 0 | CH₃CN | 2 | D4 | 86 | 313.0 |
| 61 | 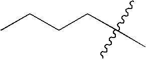 | i-Pr | C═O | 0 | CH₃CN | 2 | D4 | 55 | 305.2 |
| 62 | 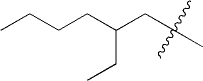 | i-Pr | C═O | 0 | acetone | 4 | D1 | 26 | 361.3 |
| 63 | 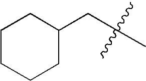 | i-Pr | C═O | 0 | CH₃CN | 2 | D4 | 53 | 344.8 |
| 64 | 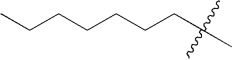 | i-Pr | C═O | 0 | CH₃CN | 2 | D4 | 51 | 346.9 |
| 65 | 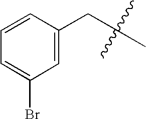 | i-Pr | C═O | 0 | CH₃CN | 2 | D4 | 61 | 417.1 |
| 66 | 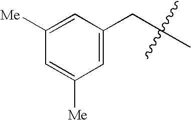 | i-Pr | C═O | 0 | CH₃CN | 2 | D4 | 72 | 367.0 |
| 67 | 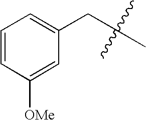 | i-Pr | C═O | 0 | CH₃CN | 2 | D4 | 72 | 368.8 |
| 68 | 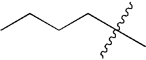 | i-Pr | CH₂ | 0 | CH₃CN | 2 | D4 | 41 | 291.1 |
| 69 | 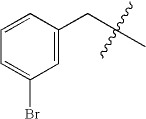 | i-Pr | CH₂ | 0 | CH₃CN | 2 | D4 | 25 | 405.1 |
| 70 | 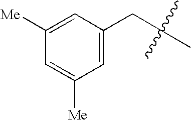 | i-Pr | CH₂ | 0 | CH₃CN | 2 | D4 | 23 | 353.2 |
| 71 | 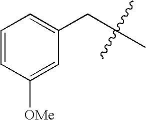 | i-Pr | CH₂ | 0 | CH₃CN | 2 | D4 | 25 | 355.0 |
| 72 | 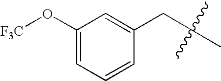 | Br | CH₂ | 1 | CH₃CN | 2 | D4 | 100 | 497.2 |
| 73 | 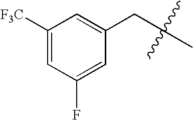 | Br | CH₂ | 1 | CH₃CN | 2 | D4 | 86 | 499 |
| 74 | 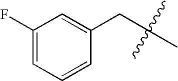 | Br | CH₂ | 1 | CH₃CN | 2 | D4 | 100 | 430.9 |
| 75 | 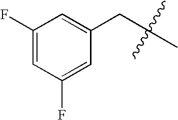 | Br | CH₂ | 1 | CH₃CN | 2 | D4 | 100 | 448.9 |
| 76 | 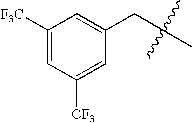 | Br | CH₂ | 1 | CH₃CN | 2 | D4 | 61 | 549.1 |
| 77 | 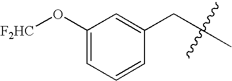 | Br | CH₂ | 1 | CH₃CN | 2 | D4 | 91 | 478.9 |
| 78 | 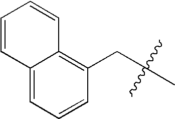 | Br | CH₂ | 1 | CH₃CN | 2 | D4 | 100 | 463.0 |
| 79 | 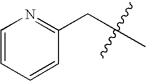 | Br | CH₂ | 1 | CH₃CN | 2 | D4 | 49 | 413.8 |
| 80 | 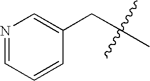 | Br | CH₂ | 1 | CH₃CN | 2 | D4 | 32 | 413.8 |
| 81 | 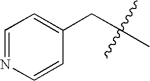 | Br | CH₂ | 1 | CH₃CN | 2 | D4 | 58 | 413.8 |
| 82 | 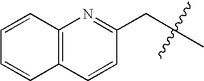 | Br | CH₂ | 1 | CH₃CN | 2 | D4 | 93 | 463.9 |
| 83 | 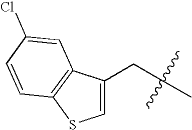 | Br | CH₂ | 1 | CH₃CN | 2 | D4 | 9 | 502.9 |
| 84 | 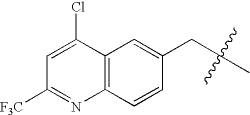 | Br | CH₂ | 1 | CH₃CN | 2 | D4 | 63 | 565.9 |
| 85 | 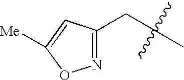 | Br | CH₂ | 1 | CH₃CN | 2 | D4 | 88 | 418.0 |
| 86 | 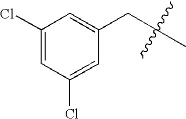 | Br | CH₂ | 1 | CH₃CN | 2 | D4 | 46 | 480.7 |
| 87 | 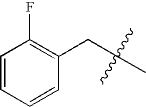 | Br | CH₂ | 1 | CH₃CN | 2 | D4 | 38 | 430.9 |
| 88 | 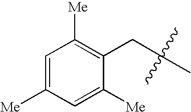 | Br | CH₂ | 1 | CH₃CN | 2 | D4 | 76 | 454.9 |
| 89 | 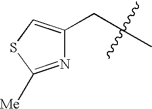 | Br | CH₂ | 1 | CH₃CN | 2 | D4 | 46 | 433.9 |
| 90 | 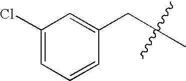 | Br | CH₂ | 1 | CH₃CN | 2 | D4 | 94 | 446.8 |
| 91 | 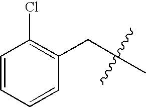 | Br | CH₂ | 1 | CH₃CN | 2 | D4 | 97 | 446.8 |
| 92 | 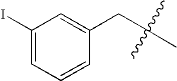 | Br | CH₂ | 1 | CH₃CN | 2 | D4 | 30 | 538.9 |
| 93 | 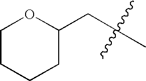 | Br | CH₂ | 1 | CH₃CN | 2 | D4 | 76 | 421.0 |
| 94 | 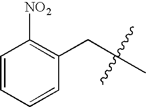 | Br | CH₂ | 1 | CH₃CN | 2 | D4 | 36 | 457.9 |
| 95 | 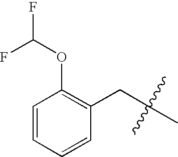 | Br | CH₂ | 1 | CH₃CN | 2 | D4 | 17 | 478.9 |
| 96 | 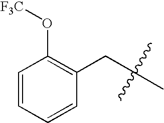 | Br | CH₂ | 1 | CH₃CN | 2 | D4 | 28 | 497.2 |
| 97 | 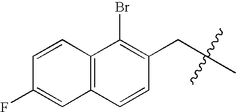 | Br | CH₂ | 1 | CH₃CN | 2 | D4 | 29 | 559.0 |
| 98 | 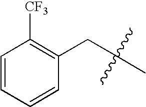 | Br | CH₂ | 1 | CH₃CN | 2 | D4 | 20 | 481.0 |
| 99 | 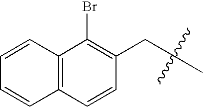 | Br | CH₂ | 1 | CH₃CN | 2 | D4 | 25 | 541.0 |
| 100 | 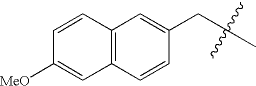 | Br | CH₂ | 1 | CH₃CN | 2 | D4 | 41 | 493.0 |

¹MS result obtained on a Perkin-Elmer API 150Ex spectrometer, using electrospray negative ion mode.

Example compounds 101 to 104 and 106 to 110 are the following:
101: 3-[3,5-dibromo-4-(3-ethoxybenzyloxy)phenyl]propionic acid
102: 3-[3,5-dibromo-4-(3-propyloxybenzyloxy)phenyl]propionic acid
103: 3-[3,5-dibromo-4-(3-butyloxybenzyloxy)phenyl]propionic acid
104: 3-[3,5-dibromo-4-(3-aminobenzyloxy)phenyl]propionic acid
106: 3-[3,5-dibromo-4-(3-diethylaminebenzyloxy)phenyl]propionic acid
107: N-[3,5-Dibromo-4-(3-bromobenzyloxy)phenyl]oxamic acid
108: N-[3,5-Dibromo-4-(2-methylnaphthyloxy)phenyl]oxamic acid
109: 3-[3-bromo-5-methoxy-4-(3-bromobenzyloxy)phenyl]propionic acid
110: 3-[3-bromo-5-methoxy-4-(2-methylnaphthyloxy)phenyl]propionic acid

Example compounds 111 to 121 are shown in Table 3:

TABLE 3

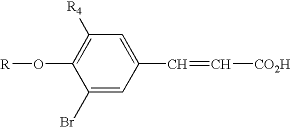

| | | | Yield | |
|---|---|---|---|---|
| Example | R | R₄ | (%) | MS¹ |
| 111 | 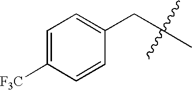 | Br | 35 | 478.9 |
| 112 | 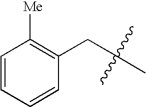 | Br | 34 | 424.9 |
| 113 | 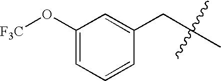 | Br | 58 | 495.1 |
| 114 | 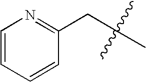 | Br | 28 | 412.0 |
| 115 | 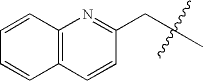 | Br | 10 | 461.8 |
| 116 | 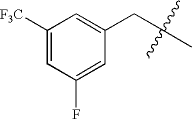 | Br | 53 | 497.2 |
| 117 | 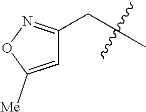 | Br | 62 | 415.9 |
| 118 | 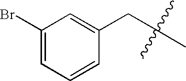 | Br | 47 | 488.8 |
| 119 | 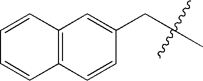 | Br | 10 | 460.9 |
| 120 | 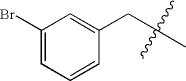 | OMe | 19 | 442.1 |
| 121 | 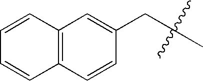 | OMe | 6 | 413.3 |

The synthesis of the Example compounds 1 to 104 and 106 to 121 is described in detail at pages 34 to 53 of WO01/36365 where the same compound numbering is used. In addition, an alternative synthesis of the compounds of Examples 95 and 113 is described here. The synthesis of example compounds 122 to 126 is described below.

### Abbreviations

BOC: tert-butyloxy
SPE: Solid Phase Extraction
SCX: benzenesulphonic acid silane, strong cation exchanger

### Alternative Synthesis for Examples 95 and 113

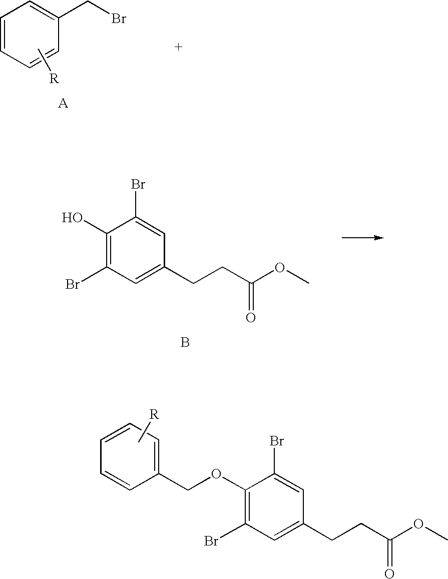

B (1 eq) was dissolved in solvent (typically acetone or acetonitrile). K₂CO₃ (4 eq) was added and mixture was stirred at room temperature for 15 min. A (1.1-1.2 eq) was added and reaction was then heated in Personal Chemistry Emrys Optimizer for 900 sec (15 min) at 120-140° C. (hold time ON, NORMAL abs, 25 sec pre-stirring). In case of incomplete reaction a second round of heating for 900 sec was applied.

The crude reaction mixture was diluted with DCM, washed with NH₄Cl(_{aq}) several times (until gas evolution ceased) and then the phases were separated on an SPE Phase Separator. The organic phase is collected and evaporated in vacuo or via N₂-stream. The crude product was purified on silica column and then used in next step. Example 95 was made according to this alternative method in 71% yield. Example 113 was made according to this alternative method in 82% yield.

### Example 122

### 3-{3,5-Dibromo-4-[2-(1H-Indol-3-yl)Ethoxy]Phenyl}Propanoic Acid

N—BOC-3-(2-bromoethyl)indole was prepared from 2-(2-bromoethyl)indole and di-tert butyldicarbonate using standard conditions.

To a solution of 3-(3,5-dibromo-4-hydroxy-phenyl)propionic acid methyl ester (12 mg, 0.035 mmol) in dry acetonitrile (0.25 mL) was added potassium carbonate (15 mg, 0.11 mmol) and the resulting mixture was stirred at room temperature. After 15 minutes a solution of N—BOC-3-(2-bromoethyl)indole (22.7 mg, 0.07 mmol) in 0.25 mL dry acetonitrile was added. The mixture was heated at 80° C. over night. After cooling down to room temperature, the mixture was filtered through a silica SPE column (500 mg/3 mL), eluting with n-heptane/ethyl acetate 3:1 (3 mL). After concentration in vacuo the residue was dissolved in tetrahydrofuran (0.25 mL) and lithium hydroxide (1 N in water, 0.25 mL) was added. The mixture was stirred over night and then neutralised on an SCX SPE column (500 mg/3 mL), using methanol as eluent. After evaporation of the solvents, the residue was dissolved in trifluoroacetic acid (10%) in dichloromethane. After four hours at room temperature, the mixture was concentrated and the residue was dissolved in methanol (0.25 mL) after which pH was adjusted to 5 with triethylamine in methanol. The mixture was again concentrated in vacuo and the product was redissolved in a minimum amount of methanol. It was then purified on an SPE-C18 column (500 mg/3 mL), using acetonitrile/H₂O 1:1 as eluent to give 12.9 mg 3-{3,5-dibromo-4-[2-(1H-indol-3-yl)ethoxy]phenyl}propanoic acid (70%) as the triethylamine salt. MS: m/z 466.0 (M⁺−1)

### Example 123

### 3-[4-(Biphenyl-2-ylmethoxy)-3,5-Dibromophenyl]Propanoic Acid

To a solution of 3-(3,5-dibromo-4-hydroxy-phenyl)propionic acid methyl ester (12 mg, 0.035 mmol) in dry acetonitrile (0.25 mL) was added potassium carbonate (15 mg, 0.11 mmol) and the resulting mixture was stirred at room temperature. After 15 minutes a solution of 2-phenylbenzyl bromide (12.8 mg, 0.07 mmol) in 0.25 mL dry acetonitrile was added. The mixture was heated at 80°0 C. over night. After cooling down to room temperature, the mixture was filtered through a silica SPE column (500 mg/3 mL), eluting with n-heptane/ethyl acetate 3:1 (3 mL). After concentration in vacuo the residue was dissolved in tetrahydrofuran (0.25 mL) and lithium hydroxide (1 N in water, 0.25 mL) was added. The mixture was stirred over night and then neutralised on an SCX SPE column (500 mg/3 mL), using methanol as eluent. After evaporation of the solvents, the residue was purified on a silica SPE column (500 mg/3 mL) with dichloromethane/methanol 9:1 as eluent giving 12.5 mg 3-[4-biphenyl-2-yl-methoxy)-3,5-dibromo-phenyl]-propionic acid (73%). MS: m/z 488.8 (M⁺−1)

### Example 124

### 3-{3,5-Dibromo-4-[(3-Bromobenzyl)Oxy]Phenyl}-2-Hydroxypropanoic Acid

To a solution of 2-hydroxy-3-(4-hydroxy-phenyl)-propionic acid (4 g, 21.95 mmol) in methanol (50 mL) was added HCl (1.5 mL). The reaction mixture was stirred at room temperature over night. The solvent was evaporated and diluted with ethyl acetate (160 mL) and which was washed with aqueous solution of sodium bicarbonate (2×20 mL). The organic phase was dried over MgSO₄ and solvent was removed. The crude reaction mixture was filtered through silica gel to give compound 2-hydroxy-3-(4-hydroxy-phenyl)-propionic acid methyl ester 3.45 g (80%).

To a mixture of 2-hydroxy-3-(4-hydroxy-phenyl)-propionic acid methyl ester (3.45 g, 17.58 mmol) in acetic acid (85 mL) and 4-5 drops of water was added sodium acetate (7.18 g, 52.8 mmol), followed by drop-wise addition of bromine (8.43 g, 52.8 mmol) in acetic acid (10 mL). The reaction mixture was stirred at room temperature for over night under dark. The reaction mixture was treated with aqueous solution of Na₂S₂O₃ and concentrated. The mixture was dissolved in ethyl acetate and washed with H₂O. The solvent was removed and the crude reaction mixture was purified on (silica gel, n-heptane/ethyl acetate, gradient elution from 100 to 40% n-heptane). This gave 3-(3,5-dibromo-4-hydroxy-phenyl)-2-hydroxy-propionic acid methyl ester (5.2 g) in 64%.

A mixture of the 3-(3,5-dibromo-4-hydroxy-phenyl)-2-hydroxy-propionic acid methyl ester (4.2 g, 11.86 mmol) and potassium carbonate (1.88 mg, 13.02 mmol) in acetonitrile (220 mL) was stirred at room temperature. After 10 minutes the m-bromo benzyl bromide (3.26 g, 13.04 mmol) was added. The reaction mixture was heated at reflux over night, cooled to room temperature and concentrated in vacuo. Diethyl ether was added to the residue, the solution filtered on silica and the resulting filtrate concentrated. The residue was dissolved in methanol 200 mL. An aqueous solution of sodium hydroxide (50 mL, 1 N) was added dropwise and the reaction mixture was stirred at room temperature for 16 hours, and acidified with hydrochloric acid (1 N). The precipitate formed was collected and dried. The crude reaction mixture was purified by HPLC (C8 column, 35% acetonitrile in aqueous ammonium acetate buffer) gave 3-[3,5-dibromo-4-(3-bromo-benzyloxy)-phenyl]-2-hydroxy-propionic acid 2.96 g (49%). MS: m/z 507 (M⁺−1)

### Example 125

### 2-Chloro-3-{3,5-Dibromo-4-[(3-Bromobenzyl)Oxy]Phenyl}Propanoic Acid

To a suspension of 4-amino-phenol (12 g, 0.11 mol) and concentrated HCl (25 mL) in acetone (175 mL), sodium nitrite (8.28 g, 0.12 mol) in H₂O (25 mL) was added at −40° C. The dark mixture was stirred for 45 minutes at −10° C. and the temperature was raised to +10° C. Methyl acrylate (50 mL, 0.56 mol) was added in the reaction mixture and heated to 30° C. and then copper (I) iodide (0.5 g, 2.6 mmol) was added portion wise. The temperature was maintained between 30° C. to 32° C. during the addition copper (I) iodide. The dark suspension was stirred at 31° C. for 30 minutes. The reaction mixture was stirred at room temperature over night. The solvent was removed at reduced pressure and the crude reaction mixture was dissolved in dichloro methane (200 mL). The organic layer was washed with water (250 mL). The aqueous layer was washed with dichloro methane (2×200 mL). The combined organic layer was washed with water (3×200 mL) and brine (250 mL). The organic layer was dried over MgSO₄ and filtered and the filtrate was evaporated at reduced pressure. The crude reaction mixture was purified by HPLC to give desired compound 2-chloro-3-(4-hydroxy-phenyl)-propionic acid methyl ester.

To a mixture of 2-chloro-3-(4-hydroxy-phenyl)-propionic acid methyl ester (100 mg, 0.47 mmol), acetic acid (4.5 mL) and 4-5 drops of water was added sodium acetate (127 mg, 0.932 mmol), followed by drop-wise addition of bromine (149 mg, 0.932 mmol) in acetic acid (1 mL). The reaction mixture was stirred at room temperature for 2 hours. The reaction mixture was treated with aqueous solution of Na₂S₂O₃ and concentrated. The mixture was dissolved in ethyl acetate (10 mL) and washed with H₂O (2×10 mL). The solvent was removed to give 2-chloro-3-(3,5-dibromo-4-hydroxy-phenyl)-propionic acid methyl ester 160 mg (92%) and which was used directly in the next step without any further purification.

A mixture of the 2-chloro-3-(3,5-dibromo-4-hydroxy-phenyl)-propionic acid methyl ester (130 mg, 0.35 mmol) and potassium carbonate (48 mg, 0.35 mmol) in acetone (15 mL) was stirred at room temperature. After 10 minutes of stirring the m-bromo benzyl bromide (87 mg, 0.35 mmol) was added. The reaction mixture was heated at reflux over night, cooled to room temperature and concentrated in vacuo. Diethyl ether was added to the residue, the solution filtered through silica gel and the resulting filtrate concentrated. The residue was dissolved in THF (1 mL) and methanol 2 mL. An aqueous solution of sodium hydroxide (0.45 mL, 1 N) was added dropwise and the reaction mixture was stirred at room temperature for 16 hours, and acidified with hydrochloric acid (1 N). The precipitate formed was collected and dried. The crude reaction mixture was purified by HPLC (40-70% acetonitrile in H₂O) gave desired 2-chloro-3-[3,5-dibromo-4-(3-bromo-benzyloxy)-phenyl]-propionic acid 94 mg (51%). MS: m/z 526.0 (M⁺−1)

### Example 126

### 3-[3,5-Dibromo-4-({2-[(E)-2-Pyridin-4-ylvinyl]Benzyl}Oxy)Phenyl] Propanoic Acid

3-[3, 5-Dibromo-4-(2-iodo-benzyloxy)-phenyl] propionic acid methyl ester was prepared from methyl 3-(3,5-dibromo-4-hydroxyphenyl)propionate and 2-iodobenzyl bromide in a procedure analogous to the preparation of Examples 122 and 123.

To a solution of 3-[3,5-dibromo-4-(2-iodo-benzyloxy)-phenyl] propionic acid methyl ester (25 mg, 0.045 mmol) in dry N,N-dimethylformamide (0.25 mL) was added a solution of 4-vinylpyridine (23.5 mg, 0.23 mmol) in dry N,N-dimethylformamide (0.25 mL) followed by triethylamine (0.031 mL, 0.23 mmol), palladium(II) acetate (1.0 mg, 0.0045 mmol) and tetrabutylammonium chloride (13 mg, 0.045 mmol). The resulting mixture was stirred at 100° C. over night and was subsequently filtered through a celite plug. After evaporation of the solvents in vacuo, the residue was purified on a silica SPE column (500 mg/3 mL) eluting with a gradient mixture starting with n-heptane/ethyl acetate 99:1. The product containing fractions were collected and concentrated and the residue was subsequently dissolved in tetrahydrofuran (0.25 mL). LiOH (_{aq}) (1 M, 0.25 mL) was added and the resulting mixture was stirred at room temperature over night. The mixture was then neutralised on an SCX SPE column (500 mg/3 mL) using triethylamine (10% in methanol) as eluent. After evaporation of the solvents the crude product was purified on a silica SPE column (1 g/3 mL) with a gradient starting with dichloromethane/methanol 99:1. The product containing fractions were collected and concentrated in vacuo yielding 2.1 mg 3-[3,5-dibromo-4-(2-((E)-2-pyridin-4-yl-vinyl)-benzyloxy)-phenyl)] propionic acid (9%). MS: m/z 516.2 (M³⁰ −1)

### FURTHER EXAMPLES

Example compounds 127 to 142 were prepared by methods analogous to those described above.
127: 3-(3,5-dibromo-4-{[3-(4-fluorophenoxy)benzyl]oxy}phenyl)propanoic acid
128: 3-(3,5-dibromo-4-{[3-(2-phenyl(E)vinyl)benzyl]oxy}phenyl)propanoic acid
129: 3-[3,5-dibromo-4-({3-[(E)-2-(4-methyl-1,3-thiazol-5-yl)vinyl]benzyl}oxy)phenyl] propanoic acid
130: 3-(4-{[3-(3-methylbenzyloxy)benzyl]oxy}-3,5-dibromophenyl)propanoic acid
131: 3-[3,5-dibromo-4-(2-naphthylmethoxy)phenyl]-2-hydroxypropanoic acid
132: 3,5-dibromo-4-[(3-bromobenzyl)oxy]-N-(phenylsulfonyl)benzamide
133: 3-(3,5-dibromo-4-{[3-(pent-4-en-1-yloxy)benzyl]oxy}phenyl)propanoic acid
134: {3,5-dibromo-4-[2-(5,5,8,8-tetramethyl-5,6,7,8-tetrahydronaphthalen-2-yl)-2-oxoethoxy]phenyl}propanoic acid
135: 3,5-dibromo-4-[(3-bromobenzyl)oxy]benzoic acid
136: 3-[3,5-dibromo-4-({3-[(E)-2-pyridin-4-ylvinyl]benzyl}oxy)phenyl]propanoic acid
137: 3-{3,5-dibromo-4-[2-(3-methyl-1-benzothien-2-yl)-2-oxoethoxy]phenyl}propanoic acid
138: 3-{3-bromo-4-[(3-bromobenzyl)oxy]-5-piperidin-1-ylphenyl}propanoic acid
139: 3-(3,5-dibromo-4-{[3-(cyclopropylmethoxy)benzyl]oxy}phenyl)propanoic acid
140: 3-[3,5-dibromo-4-({2-[(1E)-2-methyl-3-oxobut-1-en-1-yl]benzyl}oxy)phenyl] propanoic acid
141: 3-[3,5-dibromo-4-({3-[(1E)-2-methyl-3-oxobut-1-en-1-yl]benzyloxy)phenyl] propanoic acid
142: 3,5-dibromo-O-(3-bromobenzyl)tyrosine

Some specific examples of those are now described:

### Example 127

### 3-(3,5-Dibromo-4-{[3-(4-Fluorophenoxy)Benzyl]Oxy}Phenyl)Propanoic Acid

### Example 137

### 3-{3,5-Dibromo-4-[2-(3-Methyl-1-Benzothien-2-yl)-2-Oxoethoxy]Phenyl}Propanoic Acid, and

### Example 134

### {3,5-Dibromo-4-[2-(5,5,8,8-Tetramethyl-5,6,7,8-Tetrahydronaphthalen-2-yl)-2-Oxoethoxy]Phenyl}Propanoic Acid

To a solution of methyl 3-(3,5-dibromo-4-hydroxyphenyl)propionate (12 mg, 0.035 mmol) in dry acetonitrile (0.25 mL) was added potassium carbonate (15 mg, 0.11 mmol) and the resulting mixture was stirred at room temperature. After 15 minutes a solution of the appropriate halide (0.07 mmol) in 0.25 mL dry acetonitrile was added followed by sodium iodide (1 mg, 0.007 mmol) when the halide is a chloride. The mixture was heated at 80° C. over night. After cooling down to room temperature, the mixture was filtered through a silica SPE column (500 mg/3 mL), eluting with n-heptane/ethyl acetate 3:1 (3 mL). After concentration in vacuo the residue was dissolved in tetrahydrofuran (0.25 mL) and lithium hydroxide (1 N in water, 0.25 mL) was added. The mixture was stirred over night and then neutralised on an SCX SPE column (500 mg/3 mL), using methanol as eluent. After evaporation of the solvents, the residue was purified on a silica SPE column (500 mg/3 mL) with dichloromethane/methanol as eluent giving the final product.

### Yields

Example 127: KB003818: 12.4 mg (67%); MS: m/z 523.0 (M³⁰ −1)
Example 137: 6.7 mg (36%); MS: m/z 511.0 (M³⁰ −1)
Example 134: 20.4 mg (100%); MS: m/z 551.2 (M³⁰ −1)

### Example 139

### 3-(3,5-Dibromo-4-{[3-(Cyclopropylmethoxy)Benzyl]Oxy}Phenyl) Propanoic Acid and

### Example 133

### 3-(3,5-Dibromo-4-{[3-(Pent-4-en-1-yloxy)Benzyl]Oxy}Phenyl)Propanoic Acid

To a solution of methyl 3-[3,5-dibromo-4-(3-hydroxyphenyl)]propionate (15 mg, 0.032 mmol) in dry acetonitrile (0.25 mL) was added potassium carbonate (18 mg, 0.13 mmol) and the resulting mixture was stirred at room temperature. After 15 minutes a solution of the appropriate bromide (0.13 mmol) in 0.25 mL dry acetonitrile was added. The mixture was heated at 80° C. over night. After cooling down to room temperature, the mixture was filtered through a silica SPE column (400 mg/2 mL), eluting with n-heptane/ethyl acetate 3:1 (3 mL). After concentration in vacuo the residue was dissolved in tetrahydrofuran (0.25 mL) and lithium hydroxide (1 N in water, 0.25 mL) was added. The mixture was stirred over night and then neutralised on an SCX SPE column (400 mg/2 mL), using methanol as eluent. After evaporation of the solvents, the residue was purified on a silica SPE column (400 mg/2 mL) with dichloromethane/methanol 9:1 as eluent giving the final product.

### Yields

Example 139: 9.1 mg (53%); MS: m/z 541.0 (M³⁰ −1)
Example 133: 9.1 mg (52%); MS: m/z 497.2 (M³⁰ −1)

### Example 128

### 3-(3,5-Dibromo-4-{[3-(2-Phenyl(E)Vinyl)Benzyl]Oxy}Phenyl)Propanoic Acid

### Example 141

### 3-[3,5-Dibromo-4-({3-[(1E-2-Methyl-3-Oxobut-1-en-1-yl]Benzyloxy) Phenyl] Propanoic Acid

### Example 136

### 3-[3,5-Dibromo-4-({3-[(E)-2-Pyridin-4-ylvinyl]Benzyl}Oxy)Phenyl] Propanoic Acid and

### Example 129

### 3-[3,5-Dibromo-4-({3-[(E)-2-(4-Methyl-1,3-Thiazol-5-yl)Vinyl]Benzyl}Oxy) Phenyl] Propanoic Acid

Methyl 3-[3,5-dibromo-4-(3-iodophenyl)]propionate and was prepared from methyl 3-(3,5-dibromo-4-hydroxyphenyl)propionate and 3-iodobenzyl bromide in a procedure analogous to the preparation of Example 130, 142 and 138 as described above.

To a solution of methyl 3-[3,5-dibromo-4-(3-iodophenyl)]propionate (25 mg, 0.045 mmol) in dry N,N-dimethylformamide (0.25 mL) was added a solution of the appropiate alkene (0.23 mmol) in dry N,N-dimethylformamide (0.25 mL) followed by triethylamine (0.031 mL, 0.23 mmol), palladium(II)acetate (1.0 mg, 0.0045 mmol) and tetrabutylammonium chloride (13 mg, 0.045 mmol). The resulting mixture was stirred at 100° C. over night and was subsequently filtered through a celite plug. After evaporation of the solvents in vacuo, the residue was purified on a silica SPE column (500 mg/3 mL) eluting with a gradient mixture starting with n-heptane/ethyl acetate 99:1. The product containing fractions were collected and concentrated and the residue was subsequently dissolved in tetrahydrofuran (0.25 mL). LiOH (_{aq}) (1 M, 0.25 mL) was added and the resulting mixture was stirred at room temperature over night. The mixture was then neutralised on an SCX SPE column (500 mg/3 mL) using methanol or triethylamine (10% in methanol) as eluent. After evaporation of the solvents the crude product was purified on a silica SPE column (1 g/3 mL) with a gradient starting with dichloromethane/methanol 99:1. The product containing fractions were collected and concentrated in vacuo to give the final product.

### Yields

Example 128: 0.76 mg (3.3%); MS: m/z 514.8 (M³⁰ −1)
Example 141: 0.5 mg (2.2%); MS: m/z 495.2 (M³⁰ −1)
Example 136: 0.5 mg (2.1%); MS: m/z 516.2 (M³⁰ −1)
Example 129: 1.3 mg (5.4%); MS: m/z 536.0 (M³⁰ −1)

### Example 140

### 3-[3,5-Dibromo-4-({2-[(1E)-2-Methyl-3-Oxobut-1-en-1-yl]Benzyl}Oxy) Phenyl]Propanoic Acid

The procedure was carried out as described immediately above, but starting from methyl 3-[3,5-dibromo-4-(2-iodophenyl)]propionate (prepared in a procedure analogous to the preparation of 3-[3,5-dibromo-4-(3-iodophenyl)]propionate) and 3-methyl-3-butene-2-on (19.3 mg).

### Yield: 1.1 mg (5%). MS: n/z 495.2 (M³⁰ −1)

### Assay Example 1 AR Competition Binding Assay

Recombinant human androgen receptor (hAR) was extracted from Sf9 insect cells with buffer containing 1 mM EDTA, 20 mM K₂HPO₄, 8.7% glycerol, 20 mM Na₂MoO₄ and 12 mM MTG at 5*10⁷ cells/ml. The cell debris was removed by centrifugation and the supernatant aliquoted and stored at −70° C.

An aliquot of AR extract was thawed on ice prior to use and diluted to approximately 0.2 nM (1 to 30 dilution) in buffer (100 mM KₙHₘPO₄ pH 7.0, 1 mM EDTA, 8.7% glycerol, 20 mM Na₂MoO₄ and 1 mM DTT). The test ligands were diluted in DMSO as a dilution series of 10 concentrations in duplicate, with 1:5 dilution between each concentration. Tritiated mibolerone (³H-Mib) was used as tracer compound and diluted to 1.6 nM in 1 mM EDTA, 20 mM Na₂MoO₄, 8.7% glycerol and 1 mM DTT. To a 96-well polypropylene-plate 110 μl/well of 1.6 nM ³H-Mib, 10 μl/well test substance and 110 μl/well diluted AR was added. The plates were covered and incubated at +4° C. over night. The plates were harvested on filters to separate bound ligand from unbound ligand with a Tomtec Harvester. A prewet buffer containing 20 mM Kₙ(PO₄) pH 7.6, 1 mM EDTA, v/v 0.5% polyethyleneimin was used to equilibrate the filter before filtering the samples and washing the filters with 20 mM Kₙ(PO₄) pH 7.6, 1 mM EDTA 8 times. The filters were allowed to dry for 1 hour at +65° C. A scintillating wax was melted upon the filter and the radioactivity retained on the filter was measured in a Wallac Microbeta scintillation counter.

The affinity to AR was evaluated by a non-linear four-parameter logisitic model: b=(bmax−bmin)/(1+(IC50/I)Ŝ)+bmin, where bmax=total concentration of binding sites, bmin=non-specific binding, I=added concentration of binding inhibitor, IC50=concentration of binding inhibitor at half-maximal binding and S=slope factor.

### Assay Example 2 AR Transactivation Assay

The agonist and antagonist properties of compounds were determined using a cell-based system expressing stably integrated androgen receptor and an androgen responsive reporter gene. CV-1 cells (kidney fibroblasts) stably expressing CMV-hAR and alkaline phosphatase (ALP) driven by an MMTV promoter containing an androgen response element were cultured in Dulbecco's Modified Eagle Medium (DMEM), low glucose supplemented with 10% fetal bovin serum, 1% L-glutamine, and 0.7% Hygromycine B. The stably integrated cells (ARAF) were trypsinized and resuspended in Opti-MEM 1 supplemented with 2% fetal bovine serum, 1% L-Glutamine, 50 μg/ml Gentamicine and 1% Pen/Strep. The cells were counted in a Birch chamber and diluted to a concentration of 100 000 cells /ml. The cells were then seeded out in 384 plates, 5000 cells/well in 50μl seeding media and incubated overnight in 37 C., 5% CO₂.

The next day, the seeding medium was removed from the cells and 20 μl induction media (Opti-MEM 1 supplemented with 1% L-Glutamine, 50 μg/ml Gentamicine and 1% Pen/Strep) +/−0.1 nM Mibolerone was added to the wells. 10 μl of test compound diluted in induction media was then added to the wells. The cells were incubated 48 hr in 37 C., 5% CO₂.

After 48 hr 5 μl of cell medium was added to white 384 plates with 100 μl of ALP substrate buffer. The plates were incubated in 37 C. for 20 minutes followed by incubation at room temperature for 10 minutes before each well was read in a μBETA machine. Agonist activity was calculated from the alkaline phosphatase activity induced in the absence of Mibolerone and compared to standard activation curve generated by Mibolerone alone. Antagonist activity was calculated from the decrease in ALP activity in the presence of 0.1 nM Mibolerone. EC50 and IC50 values were calculated by using a non-linear four-parameter fit as described above.

Compounds of the invention were found to exhibit binding affinities to the AR receptor in the range of from 40 nM to 10000 nM.

Agonist activity of the compounds of the invention may be determined in an analogous fashion.

Other assays to determine androgen receptor mediated activity of the test compounds include modulation of endogenous AR mediated transcription in cell culture systems; modulation of androgen responsive tissue effects in rodents; identification of receptor surface conformation changes; and binding specificity to AR versus other nuclear receptors.

1. A compound of formula (I) or a pharmaceutically acceptable ester, amide, solvate or salt thereof, including a salt of such an ester or amide, and a solvate of such an ester, amide or salt, for use in the treatment or prophylaxis of a condition mediated by an androgen receptor, 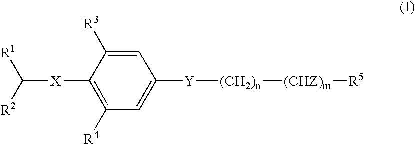
wherein:
R¹ is selected from C₅₋₁₀ aryl, C(O)—C₅₋₁₀ aryl, C(O)—C₃₋₈ heterocyclyl, C₅₋₁₀ aryl-C₁₋₂ alkyl, C₃₋₁₀ heterocyclyl, C₃₋₁₀ heterocyclyl-C₁₋₂ alkyl, C₃₋₁₅ alkyl, C₄₋₁₅ alkenyl, C₃₋₁₅ alkynyl, C₃₋₁₀ cycloalkyl and C₃₋₁₀ cycloalkyl C₁₋₂ alkyl, said alkyl, alkenyl and alkynyl groups or portions of groups optionally being substituted with, where applicable, 1 to 3 groups R^{a} which may be the same or different; said heterocyclyl and cycloalkyl groups or portions of groups optionally being substituted with, where applicable, 1 to 3 groups R^{a′} which may be the same or different; said aryl groups or portions of groups optionally being substituted with, where applicable, 1 to 4 groups R^{a″} which may be the same or different;
R² is selected from hydrogen, C₁₋₄ alkyl, C₂₋₄ alkenyl, C₂₋₄ alkynyl and C₁₋₄ alkoxy;
or R¹ and R² together with the carbon atom to which they are both attached form a C₄₋₈ cycloalkyl, C₄₋₈ cycloalkenyl, a saturated or partially saturated C₃₋₁₀ heterocyclyl, optionally substituted with, where applicable, 1 to 3 groups R^{a′} which may be the same or different;
X is selected from CH₂, oxygen, sulfur, sulfoxide, sulfone, selenium, tellurium, disulfide, and a group of formula —N(R^{c})—;
R³ and R⁴ are independently selected from hydrogen, halogen, C₁₋₄ alkyl, C₃₋₇ cycloalkyl, C₃₋₇ heterocyclyl, C₂₋₄ alkenyl, C₂₋₄ alkynyl, C₁₋₄ alkoxy, fluoromethyl, difluoromethyl, trifluoromethyl, fluoromethoxy, difluoromethoxy, trifluoromethoxy, methylthio, fluoromethylthio, difluoromethylthio, trifluoromethylthio, and COOR^{c};
Y is selected from bond, carbonyl, oxygen, sulphur, —CH(R^{b})—, —NHCO—, —CONH—, —NHSO₂—, —SO₂NH—, —N(R^{c})— and —CR⁶═CR⁷—;
n is selected from 0, 1, 2 and 3;
Z is selected from halogen, amino, hydroxy, mercapto, C₁₋₄ alkyl, C₂₋₄ alkenyl, C₂₋₄ alkynyl, C₁₋₄ alkoxy, fluoromethyl, difluoromethyl, trifluoromethyl, fluoromethoxy, difluoromethoxy, trifluoromethoxy and (CH₂)ₚOH, where p is an integer from 1 to 4;
m is selected from 0 and 1;
R⁵ is selected from —CO₂R^{c}, —PO(OR^{c})_{2,} —PO(OR^{c})NH₂, —SO₂OR^{c}, —COCO₂R^{c}, CONR^{c}OR^{c}, —SO₂NHR^{c}, —NHSO₂R^{c′}, —CONHSO₂R^{c′}, and —SO₂NHCOR^{c};
R⁶ and R⁷ are independently selected from hydrogen, halogen, C₁₋₄ alkyl, C₂₋₄ alkenyl, C₂₋₄ alkynyl, C₁₋₄ alkoxy, C₅₋₁₀ aryl, fluoromethyl, difluoromethyl, trifluoromethyl, fluoromethoxy, difluoromethoxy, trifluoromethoxy, and (CH₂)ₚOH, where p is an integer from 1 to 4;
^{R}^{a} is selected from halogen, C₁₋₄ alkoxy, C₅₋₁₀ aryl, fluoromethoxy, difluoromethoxy, trifluoromethoxy, methylthio, fluoromethylthio, difluoromethylthio, trifluoromethylthio, mercapto, cyano, and nitro;
R^{a′} is selected from R^{a}, fluoromethyl, difluoromethyl, trifluoromethyl, C₁₋₄ alkyl, C₃₋₁₀ heterocyclyl-C₂₋₄ alkenyl, C₅₋₁₀ aryl-C₂₋₄ alkenyl, C₃₋₁₀ heterocyclyl-C₁₋₄ alkyl and C₅₋₁₀ aryl-C₁₋₄ alkyl;
R^{a″} is selected from:
R^{a′};
C₂₋₄ alkenyl, optionally substituted with 1, 2 or 3 groups selected from C₅₋₁₀ aryl, C(O)R^{c}, C₃₋₁₀ heterocyclyl, and C₃₋₁₀ heterocyclyl substituted with C₁₋₄ alkyl;
C₂₋₈ alkenyloxy;
C₃₋₈ cycloalkyl-C₁₋₃ alkoxy, C₅₋₁₀ aryl-C₁₋₃ alkoxy, and C₅₋₁₀ aryloxy, said C₃₋₈ cycloalkyl-C₁₋₃ alkoxy, C₅₋₁₀ aryl-C₁₋₃ alkoxy or C₅₋₁₀ aryloxy optionally being substituted with 1, 2 or 3 groups selected from C₁₋₄ alkyl, halogen, fluoromethyl, difluoromethyl, trifluoromethyl, fluoromethoxy, difluoromethoxy, trifluoromethoxy, methylthio, mercapto, hydroxy, cyano, nitro, a group of formula —N(R^{c})₂ in which the two R^{c} groups may be the same or different but not both simultaneously hydrogen;
R^{b} is selected from hydrogen, halogen, hydroxyl, mercapto, C₁₋₄ alkyl, C₂₋₄ alkenyl, C₂₋₄ alkynyl, C₁₋₄ alkoxy, fluoromethyl, difluoromethyl, trifluoromethyl, fluoromethoxy, difluoromethoxy, trifluoromethoxy and (CH₂)ₚOH, where p is an integer from 1 to 4; and
R^{c} is selected from hydrogen, C₁₋₄ alkyl, C₂₋₄ alkenyl and C₂₋₄ alkynyl; and
R^{c} is selected from R^{c}, C₅₋₁₀ aryl and C₅₋₁₀ aryl substituted with 1, 2 or 3 groups selected from amino, hydroxy, halogen or C₁₋₄ alkyl.

2. A compound as claimed in claim 1 which is of formula (Ia) or a pharmaceutically acceptable ester, amide, solvate or salt thereof, including a salt of such an ester or amide, and a solvate of such an ester, amide or salt, for use in the treatment or prophylaxis of a condition mediated by an androgen receptor, 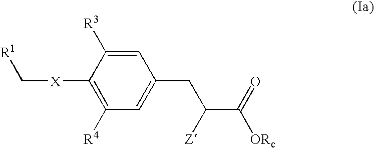
wherein:
R¹ is selected from C₆₋₁₀ aryl, C₅₋₁₀ heterocyclyl-C₁₋₂-alkyl, C₄₋₁₀ alkyl and C₅₋₇ cycloalkyl, said alkyl optionally being substituted with, where applicable, 1 to 3 groups
R^{a} which may be the same or different; said cycloalkyl optionally being substituted with, where applicable, 1 to 3 groups R^{a′} which may be the same or different; and said aryl optionally being substituted with, where applicable, 1 to 3 groups R^{a″} which may be the same or different;
X is selected from oxygen and sulfur;
R³ and R⁴ are independently selected from hydrogen, halogen, C₁₋₂ alkyl, C₁₋₂ alkoxy, fluoromethyl, difluoromethyl, trifluoromethyl, fluoromethoxy, difluoromethoxy and trifluoromethoxy;
Z′ is selected from hydrogen, halogen, hydroxyl and mercapto;
R^{a} is selected from halogen, C₅₋₁₀ aryl, fluoromethoxy, difluoromethoxy, trifluoromethoxy and nitro;
R^{a} is selected from R^{a}, fluoromethyl, difluoromethyl, trifluoromethyl, C₁₋₄ alkyl C₅₋₁₀ heterocyclyl-C₂₋₄ alkenyl, C₅₋₁₀ aryl-C₂₋₄ alkenyl, C₅₋₁₀ heterocyclyl-C₁₋₄ alkyl and C₅₋₁₀ aryl-C₁₋₄ alkyl;
R^{a″} is selected from:
R^{a′};
C₂₋₄ alkenyl, substituted with C₃₋₁₀ heterocyclyl;
C₅₋₁₀ aryloxy, optionally being substituted with 1, 2 or 3 groups selected from C₁₋₄ alkyl, halogen, fluoromethyl, difluoromethyl, trifluoromethyl, fluoromethoxy, difluoromethoxy, trifluoromethoxy, methylthio, mercapto, hydroxy, cyano, or nitro;
and
R^{c} is selected from hydrogen and C₁₋₄ alkyl.

3. A compound as claimed in claim 1 or claim 2 whereby the condition mediated by an androgen receptor is selected from the group consisting of: prostate cancer, psychological abnormalities (including mood (depression, aggression, anxiety) and cognitive function), male pattern baldness (alopecia), benign prostatic hyperplasia (BPH), amenorrhea, hypogonadism, anemia, defects in spermatogenesis, cachexia, osteoporosis, osteopenia, and muscle wasting.

4. A method for the treatment or prophylaxis of a condition in a mammal mediated by an androgen receptor, which comprises administering to the mammal a therapeutically effective amount of a compound of formula (I) as defined in claim 1 or a pharmaceutically acceptable ester, amide, solvate or salt thereof, including a salt of such an ester or amide, and a solvate of such an ester, amide or salt.

5. Use of a compound of formula (I) as defined in claim 1 or a compound of formula (Ia) as defined in claim 2, or a pharmaceutically acceptable ester, amide, solvate or salt thereof, including a salt of such an ester or amide, and a solvate of such an ester, amide or salt, for the manufacture of a medicament for the treatment or prophylaxis of a condition mediated by an androgen receptor.

6. A pharmaceutical formulation comprising a compound of formula (I) as defined in claim 1 or a compound of formula (Ia) as defined in claim 2, or a pharmaceutically acceptable ester, amide, solvate or salt thereof, including a salt of such an ester or amide, and a solvate of such an ester, amide or salt, and a pharmaceutically acceptable excipient.

7. Use of a compound as defined in claim 1 or claim 2 in labelled form as a diagnostic agent for the diagnosis of conditions associated with malfunction of the androgen receptor.

8. A method of discovering a ligand of the androgen receptor which comprising use of a compound as defined in claim 1 or claim 2 or a compound as defined in claim 1 or claim 2 in labelled form, as a reference compound.

9. A compound of formula (Ib) or a pharmaceutically acceptable ester, amide, solvate or salt thereof, including a salt of such an ester or amide, and a solvate of such an ester, amide or salt, 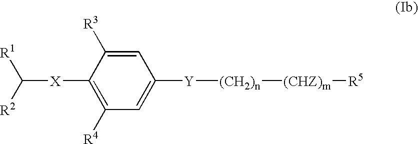
wherein:
R¹ is selected from C₅₋₁₀ aryl, C(O)—C₅₋₁₀ aryl, C(O)—C₃₋₈ heterocyclyl, C₅₋₁₀ aryl-C₁₋₂ alkyl, C₃₋₁₀ heterocyclyl, C₃₋₁₀ heterocyclyl-C₁₋₂ alkyl, C₃₋₁₅ alkyl, C₄₋₁₅ alkenyl, C₃₋₁₅ alkynyl, C₃₋₁₀ cycloalkyl and C₃₋₁₀ cycloalkyl C₁₋₂ alkyl, said alkyl, alkenyl and alkynyl optionally being substituted with, where applicable, 1 to 3 groups R^{a} which may be the same or different; said aryl-alkyl, heterocyclyl and cycloalkyl optionally being substituted with, where applicable, 1 to 3 groups R^{a′} which may be the same or different; said aryl optionally being substituted with, where applicable, 1 to 4 groups R^{a″} which may be the same or different;
R² is selected from hydrogen, C₁₋₄ alkyl, C₂₋₄ alkenyl, C₂₋₄ alkynyl and C₁₋₄ alkoxy;
or R¹ and R² together with the carbon atom to which they are both attached form a C₄₋₈ cycloalkyl, C₄₋₈ cycloalkenyl, a saturated or partially saturated C₃₋₁₀ heterocyclyl, optionally substituted with, where applicable, 1 to 3 groups R^{a′} which may be the same or different;
X is selected from CH₂, oxygen, sulfur, sulfoxide, sulfone, selenium, tellurium, disulfide, and a group of formula —N(R^{c})—;
R³ and R⁴ are independently selected from hydrogen, halogen, C₁₋₄ alkyl, C₃₋₇ cycloalkyl, C₃₋₇ heterocyclyl, C₂₋₄ alkenyl, C₂₋₄ alkynyl, C₁₋₄ alkoxy, fluoromethyl, difluoromethyl, trifluoromethyl, fluoromethoxy, difluoromethoxy, trifluoromethoxy, methylthio, fluoromethylthio, difluoromethylthio, trifluoromethylthio, and COOR^{c};
Y is selected from bond, carbonyl, oxygen, sulphur, —CH(R^{b})—, —NHCO—, —NHSO₂—, —SO₂NH—, —N(R^{c})— and —CR⁶═CR⁷—;
n is selected from 0, 1, 2 and 3;
Z is selected from halogen, amino, hydroxy, mercapto, C₁₋₄ alkyl, C₂₋₄ alkenyl, C₂₋₄ alkynyl, C₁₋₄ alkoxy, fluoromethyl, difluoromethyl, trifluoromethyl, fluoromethoxy, difluoromethoxy, trifluoromethoxy and (CH₂)^{p}OH, where p is an integer from 1 to 4;
R⁵ is selected from —CO₂R^{c}, —PO(OR^{c})_{2,} —PO(OR^{c})NH₂, —SO₂OR^{c}, —COCO₂R^{c}, CONR^{c}OR^{c}, —SO₂NHR^{c}, —NHSO₂R^{c′}, —CONHSO₂R^{c}, and — SO₂NHCOR^{c};
R⁶ and R⁷ are independently selected from hydrogen, halogen, C₁₋₄ alkyl, C₂₋₄ alkenyl, C₂₋₄ alkynyl, C₁₋₄ alkoxy, C₅₋₁₀ aryl, fluoromethyl, difluoromethyl, trifluoromethyl, fluoromethoxy, difluoromethoxy, trifluoromethoxy, and (CH₂)ₚOH, where p is an integer from 1 to 4;
R^{a} is selected from halogen, C₁₋₄ alkoxy, C₅₋₁₀ aryl, fluoromethoxy, difluoromethoxy, trifluoromethoxy, methylthio, fluoromethylthio, difluoromethylthio, trifluoromethylthio, mercapto, cyano, and nitro;
R^{a′} is selected from R^{a}, fluoromethyl, difluoromethyl, trifluoromethyl, C₁₋₄ alkyl, C₃₋₁₀ heterocyclyl-C₂₋₄ alkenyl, C₅₋₁₀ aryl-C₂₋₄ alkenyl, C₃₋₁₀ heterocyclyl-C₁₋₄ alkyl and C₅₋₁₀ aryl-C₁₋₄ alkyl;
R^{a″} is selected from:
R^{a′};
C₂₋₄ alkenyl, optionally substituted with 1, 2 or 3 groups selected from C₅₋₁₀ aryl, C(O)R^{c}, C₃₋₁₀ heterocyclyl, and C₃₋₁₀ heterocyclyl substituted with C₁₋₄ alkyl;
C₂₋₈ alkenyloxy;
C₃₋₈ cycloalkyl-C₁₋₃ alkoxy, C₅₋₁₀ aryl-C₁₋₃ alkoxy, or C₅₋₁₀ aryloxy, said C₃₋₈ cycloalkyl-C₁₋₃ alkoxy, C₅₋₁₀ aryl-C₁₋₃ alkoxy or C₅₋₁₀ aryloxy optionally being substituted with 1, 2 or 3 groups selected from C₁₋₄ alkyl, halogen, fluoromethyl, difluoromethyl, trifluoromethyl, fluoromethoxy, difluoromethoxy, trifluoromethoxy, methylthio, mercapto, hydroxy, cyano, nitro, a group of formula —N(R^{c})₂ in which the two R^{c} groups may be the same or different but not both simultaneously hydrogen;
R^{b} is selected from hydrogen, halogen, hydroxyl, mercapto, C₁₋₄ alkyl, C₂₋₄ alkenyl, C₂₋₄ alkynyl, C₁₋₄ alkoxy, fluoromethyl, difluoromethyl, trifluoromethyl, fluoromethoxy, difluoromethoxy, trifluoromethoxy and (CH₂)ₚOH, where p is an integer from 1 to 4; and
R^{c} is selected from hydrogen, C₁₋₄ alkyl, C₂₋₄ alkenyl and C₂₋₄ alkynyl;
R^{c} is selected from R^{c}, C₅₋₁₀ aryl or C₅₋₁₀ aryl substituted with amino, hydroxyl, halogen or C₁₋₄ alkyl; and
m is 1; or
simultaneously m is 0 or 1 and R³ is C₃₋₇ heterocyclyl; or
simultaneously Y is bond, m is 0, n is 0 and R⁵ is —CO₂R^{c}.

10. A compound of formula (Ic) or a pharmaceutically acceptable ester, amide, solvate or salt thereof, including a salt of such an ester or amide, and a solvate of such an ester, amide or salt, 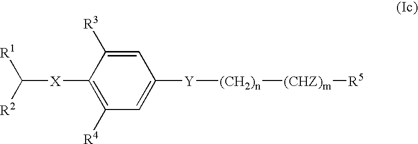
wherein:
R¹ is selected from C₅₋₁₀ aryl, C(O)—C₅₋₁₀ aryl, C(O)—C₃₋₈ heterocyclyl or C₅₋₁₀ heterocyclyl-C₁₋₂ alkyl,
said C(O)—C₅₋₁₀ aryl, C(O)—C₃₋₈ heterocyclyl or C₅₋₁₀ heterocyclyl-C₁₋₂ alkyl optionally being substituted with, where applicable, 1 to 3 groups R^{a′} which may be the same or different;
said C₅₋₁₀ aryl being substituted with a group selected from:
C₅₋₁₀ aryl, fluoromethoxy, difluoromethoxy, trifluoromethoxy, mercapto, fluoromethyl, difluoromethyl, and C₃₋₁₀ heterocyclyl-C₂₋₄ alkenyl;
C₂₋₄ alkenyl, substituted with 1, 2 or 3 groups selected from C₅₋₁₀ aryl, C(O)R^{c}, C₃₋₁₀ heterocyclyl, and C₃₋₁₀ heterocyclyl substituted with C₁₋₄ alkyl;
C₂₋₈ alkenyloxy;
C₃₋₈ cycloalkyl-C₁₋₃ alkoxy, C₅₋₁₀ aryl-C₁₋₃ alkoxy, or C₅₋₁₀ aryloxy, said C₃₋₈ cycloalkyl-C₁₋₃ alkoxy, C₅₋₁₀ aryl-C₁₋₃ alkoxy or C₅₋₁₀ aryloxy optionally being substituted with 1, 2 or 3 groups selected from C₁₋₄ alkyl, halogen, fluoromethyl, difluoromethyl, trifluoromethyl, fluoromethoxy, difluoromethoxy, trifluoromethoxy, methylthio, mercapto, hydroxy, cyano, nitro, a group of formula —N(R^{c})₂ in which the two R^{c} groups may be the same or different but not both simultaneously hydrogen;
said aryl optionally also substituted with, where applicable, 1 to 2 groups R^{a′} which may be the same or different,
R² is selected from hydrogen, C₁₋₄ alkyl, C₂₋₄ alkenyl, C₂₋₄ alkynyl and C₁₋₄ alkoxy;
X is selected from CH₂, oxygen, sulfur, sulfoxide, sulfone, selenium, tellurium, disulfide, and a group of formula —N(R^{c})—;
R³ and R⁴ are independently selected from hydrogen, halogen, C₁₋₄ alkyl, C₃₋₇ cycloalkyl, C₃₋₇ heterocyclyl, C₂₋₄ alkenyl, C₂₋₄ alkynyl, C₁₋₄ alkoxy, fluoromethyl, difluoromethyl, trifluoromethyl, fluoromethoxy, difluoromethoxy, trifluoromethoxy, methylthio, fluoromethylthio, difluoromethylthio, trifluoromethylthio, and COOR^{c};
Y is selected from bond, carbonyl, oxygen, sulphur, —CH(R^{b})—, —NHCO—, —NHSO₂—, —SO₂NH—, —N(R^{c})— and —CR⁶═CR⁷—;
n is selected from 0, 1, 2 and 3;
Z is selected from halogen, amino, hydroxy, mercapto, C₁₋₄ alkyl, C₂₋₄ alkenyl, C₂₋₄ alkynyl, C₁₋₄ alkoxy, fluoromethyl, difluoromethyl, trifluoromethyl, fluoromethoxy, difluoromethoxy, trifluoromethoxy and (CH₂)ₚOH, where p is an integer from 1 to 4;
m is selected from 0 and 1;
R⁵ is selected from —CO₂R^{c}, —PO(OR^{c})₂, —PO(OR^{c})NH₂, —SO₂OR^{c}, —COCO₂R^{c}, CONR^{c}OR^{c}, —SO₂NHR^{c}, —NHSO₂R^{c′}, —CONHSO₂R^{c}, and — SO₂NHCOR^{c};
R⁶ and R⁷ are independently selected from hydrogen, halogen, C₁₋₄ alkyl, C₂₋₄ alkenyl, C₂₋₄ alkynyl, C₁₋₄ alkoxy, C₅₋₁₀ aryl, fluoromethyl, difluoromethyl, trifluoromethyl, fluoromethoxy, difluoromethoxy, trifluoromethoxy, and (CH₂)ₚOH, where p is an integer from 1 to 4;
R^{a} is selected from halogen, C₁₋₄ alkoxy, C₅₋₁₀ aryl, fluoromethoxy, difluoromethoxy, trifluoromethoxy, methylthio, fluoromethylthio, difluoromethylthio, trifluoromethylthio, mercapto, cyano, and nitro;
R^{a′} is selected from R^{a}, fluoromethyl, difluoromethyl, trifluoromethyl, C₁₋₄ alkyl, and C₃₋₁₀ heterocyclyl-C₂₋₄ alkenyl;
R^{b} is selected from hydrogen, halogen, hydroxyl, mercapto, C₁₋₄ alkyl, C₂₋₄ alkenyl, C₂₋₄ alkynyl, C₁₋₄ alkoxy, fluoromethyl, difluoromethyl, trifluoromethyl, fluoromethoxy, difluoromethoxy, trifluoromethoxy and (CH₂)ₚOH, where p is an integer from 1 to 4; and
R^{c} is selected from hydrogen, C₁₋₄ alkyl, C₂₋₄ alkenyl and C₂₋₄ alkynyl; and
R^{c} is selected from R^{c}, C₅₋₁₀ aryl or C₅₋₁₀ aryl substituted with amino, hydroxyl, halogen or C₁₋₄ alkyl.

11. A compound as claimed in claim 9 or 10 for use as a medicament.

12. A method for preparing a compound of formula (Ib) as described in claim 9 or a compound of formula (Ic) as described in claim 10 comprising a step of adding a compound of formula (II) 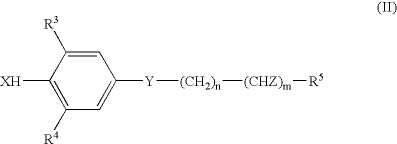
wherein X, R³, R⁴, Y, n, Z, m and R⁵ are as defined in claim 9 or 10, with a compound of formula (III) 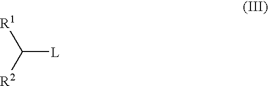
wherein R¹ and R² are as defined in, as appropriate, claim 9 or 10 and L is a suitable leaving group, optionally in the presence of a suitable base.
